SEQUENCE LISTING (14 sequences)

SEQ ID NO: 1 - protein, 447 aa, Artificial Sequence
  Description of Artificial Sequence Synthetic polypeptide
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 215 aa, Artificial Sequence
  Description of Artificial Sequence Synthetic polypeptide
ETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 447 aa, Artificial Sequence
  Description of Artificial Sequence Synthetic polypeptide
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 5 aa, Artificial Sequence
  Description of Artificial Sequence Synthetic peptide
SNVIS

SEQ ID NO: 5 - protein, 17 aa, Artificial Sequence
  Description of Artificial Sequence Synthetic peptide
GVIPIVDIANYAQRFKG

SEQ ID NO: 6 - protein, 11 aa, Artificial Sequence
  Description of Artificial Sequence Synthetic peptide
TLGLVLDAMDY

SEQ ID NO: 7 - protein, 12 aa, Artificial Sequence
  Description of Artificial Sequence Synthetic peptide
RASQSLGSSYLA

SEQ ID NO: 8 - protein, 7 aa, Artificial Sequence
  Description of Artificial Sequence Synthetic peptide
GASSRAP

SEQ ID NO: 9 - protein, 9 aa, Artificial Sequence
  Description of Artificial Sequence Synthetic peptide
QQYADSPIT

SEQ ID NO: 10 - protein, 120 aa, Artificial Sequence
  Description of Artificial Sequence Synthetic polypeptide
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSS

SEQ ID NO: 11 - protein, 108 aa, Artificial Sequence
  Description of Artificial Sequence Synthetic polypeptide
ETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIK

SEQ ID NO: 12 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 107 aa, Homo sapiens [sequence omitted]

SEQ ID NO: 14 - protein, 10 aa, Artificial Sequence
  Description of Artificial Sequence Synthetic peptide
DDDDDDDDDD

(19) United States
(12) Patent Application Publication
(10) Pub. No.: US 2024/0238415 A1
(43) Pub. Date: Jul. 18, 2024

(54) Treatment of Moderate-to-Severe Osteogenesis Imperfecta

(71) Applicant: Baylor College of Medicine, Houston, TX (US)

(72) Inventor: Brendan LEE, Houston, TX (US)

(73) Assignee: Baylor College of Medicine, Houston, TX (US)

(21) Appl. No.: 18/559,261

(22) PCT Filed: May 4, 2022
(86) PCT No.: PCT/US2022/027666

§ 371 Date: Nov. 6, 2023

Related U.S. Application Data
(60) Provisional application No. 63/185,967, filed on May 7, 2021.

(51) Int. Cl. A61K 39/395; A61K 31/663; A61K 38/23; A61K 38/29; A61K 47/64; A61P 19/08; C07K 16/22

(52) U.S. Cl. CPC A61K 39/3955; A61K 31/663; A61K 38/23; A61K 38/29; A61K 47/645; A61P 19/08; C07K 16/22; C07K 2317/21

(57) ABSTRACT

The present disclosure provides methods for treating and improving moderate-to-severe osteogenesis imperfecta (OI) in a subject by administering to the subject a therapeutically effective amount of an agent that binds and neutralizes transforming growth factor beta (TGFβ).

### CROSS-REFERENCE TO RELATED APPLICATION

This application claims priority from U.S. Provisional Application 63/185,967 filed on May 7, 2021. The disclosure of this priority application is incorporated herein by reference in its entirety.

### GOVERNMENT FUNDING

This invention was made with government support under Grant AR068069 awarded by the National Institutes of Health. The United States government has certain rights in the invention.

### SEQUENCE LISTING

The instant application contains a Sequence Listing which has been submitted electronically in ASCII format and is hereby incorporated by reference in its entirety. Said ASCII copy, created on May 3, 2022, is named 022548_WO026_SL.txt and is 17,875 bytes in size.

### JOINT RESEARCH AGREEMENT

This work was supported by a research agreement with Sanofi Genzyme.

### BACKGROUND OF THE INVENTION

Osteogenesis Imperfecta (OI) is a genetically and phenotypically heterogeneous Mendelian disorder of connective disorder that has an estimated prevalence of 1 in 10,000-20,000 births. The skeletal manifestations of OI include low bone mass, bone fragility, recurrent fractures, scoliosis, and bone deformities. The extraskeletal manifestations include decreased muscle mass, muscle weakness, dentinogenesis imperfecta, hearing loss, and pulmonary disease (Marini, Nat Rev Dis Primers (2017)3:17052; Marom et al., Am J Med Genet C Semin Med Genet. (2016) 172(4):367-83; Patel et al., Clin Gen. (2015) 87(2):133-40; Rossi et al., Curr Opin Pediatr. (2019) 31(6):708-15; Tam et al., Clin Gen. (2018) 94(6):502-11; DiMeglio et al. J Bone Miner Res. (2006) 21:132-40; Gatti et al., J Bone Miner Res. (2005) 20(5):758-63); Gatti et al., Calcified Tissue Int. (2013) 93(5):448-52). The management of individuals with OI typically involves a multidisciplinary approach. The mainstay therapy for OI bone fragility involves repurposing of medications that are used to treat osteoporosis (Adami et al., J Bone Miner Res. (2003)18(1):126-30; Bishop et al., Far Hum Dev. (2010) 86(11):743-6; Chevrel et al., J Bone Miner Res. (2006) 21(2):300-6; Glorieux et al., NEJM (1998) 339(14):947-52; Rauch et al., J Bone Miner Res. (2009) 24(7): 1282-9; Rauch et al., J Bone Miner Res. (2003) 18(4):610-4; Orwoll et al., J Clin Invest (2014) 124(2):491-8; Hoyer-Kuhn et al., J Musculoskelet Neuronal Interact/(2016) 16(1):24-32; Anissipour et al., J Bone Joint Surg Am. (2014) 96(3):237-43).

Bisphosphonates (BPN), a class of antiresorptive medications that decrease bone remodeling, have become the standard of care, especially in pediatric OI. In children, BPN has been shown to have beneficial effects on areal and volumetric bone mineral density (aBMD and vBMD), progression of scoliosis, quality-of-life, and in some studies, fracture incidence (Bishop et al., Lancet (2013) 382(9902): 1424-32; Rauch et al., 2003, supra; Bains et al., JBMR Plus (2019) 3(5):e10118; Rauch et al., Bone (2007) 40(2):274-80). However, given the heterogeneity of OI and the variability in the clinical study designs, the effects of BPN have been inconsistent. In adults, the benefits and the consequences of long-term treatment with bisphosphonates are less certain (Adami et al., ibid; Shi et al., Am J Ther. (2016) 23(3):e894-904). Additionally, in a randomized trial involving adults with OI, treatment with an anabolic agent, teriparatide, led to increase in aBMD and vBMD in individuals with the mild form (OI type I) but not in the moderate-to-severe forms of the disorder (OI types III and IV). Furthermore, none of these repurposed therapies address specific pathogenetic mechanism in OI, and hence, they have no effect on extraskeletal manifestations.

Therefore, there remains a significant unmet need for effective therapy targeting moderate-to-severe forms of OI.

### SUMMARY OF THE INVENTION

The present disclosure provides a method for treating osteogenesis imperfecta (OI) in a human subject in need thereof, comprising administering to the subject a therapeutically effective amount of an anti-TGFβ antibody or an antigen-binding fragment thereof, wherein the antibody or antigen-binding fragment comprises heavy chain complementarity-determining regions (CDRs) 1-3 comprising SEQ ID NOs:4-6, respectively, and light chain CDR1-3 comprising SEQ ID NOs: 7-9, respectively, wherein the therapeutic effective amount is 1-10 mg/kg (e.g., 1, 2, 3, 4, 5, 6, 7, 8, 9, or 10 mg/kg).

In some embodiments, the antibody or antigen-binding fragment comprises a heavy chain variable domain comprising SEQ ID NO: 10 and a light chain variable domain comprising SEQ ID NO:11. In some embodiments, the antibody comprises a human IgG₄ constant region and/or a human κ light chain constant region. In certain embodiments, the human IgG₄ constant region comprises a S228P mutation (Eu numbering). In particularly embodiments, the antibody comprises a heavy chain comprising SEQ ID NO:1 and a light chain comprising SEQ ID NO:2. In other particular embodiments, the antibody comprises a heavy chain comprising SEQ ID NO:3 and a light chain comprising SEQ ID NO:2.

In some embodiments, the antibody comprises a bone-targeting moiety, optionally wherein the bone-targeting moiety is a poly-arginine peptide. In some embodiments, the antibody comprises one or more poly-arginine peptides, for example, at the N-terminus, or the C-terminus, or both termini, of the heavy chain, and/or at the C-terminus of the light chain, of the antibody or antigen-binding fragment. In particular embodiments, the poly-arginine peptide is D10 (SEQ ID NO:14).

In some embodiments, the OI is moderate-to-severe OI or type IV OI. In some embodiments, the human subject is an adult patient (≥18 years of age), or a pediatric patient (<18 years of age). In some embodiments, the human subject has a mutation in a COL1A1 or COL1A2 gene, optionally wherein the mutation is a glycine substitution mutation in the COL1A1 or COL1A2 gene or a valine deletion in the COL1A2 gene.

In some embodiments, the administration improves a bone parameter selected from the group consisting of bone mineral density (BMD), bone volume density (BV/TV), total bone surface (BS), bone surface density (BS/BV), trabecular number (Tb.N), trabecular thickness (Tb.Th), trabecular spacing (Tb.Sp), and total volume (Dens TV). In further embodiments, the bone parameter is lumbar spine areal BMD (LS aBMD), optionally wherein the LS aBMD increases by at least 1-10% after the administration relative to baseline level.

In some embodiments, the administration decreases bone turnover and/or osteocyte density, optionally wherein the decreased bone turnover is indicated by a decrease in serum CTX or an increase in serum osteocalcin (OCN).

In some embodiments, the administering step is repeated every month, every two months, every three months, every six months, every nine months, or every twelve months, for example, at a dose of 4 mg/kg. The antibody or antigen-binding fragment may be administered intravenous infusion.

In some embodiments, the method further comprises administering to the subject another therapeutic, such as a bisphosphonate (e.g., alendronate, pamidronate, zoledronate, and risedronate), parathyroid hormone, calcitonin, teriparatide, or an anti-sclerostin agent.

Also provided herein are an anti-TGFβ antibody or an antigen-binding fragment thereof for use in treating osteogenesis imperfecta in the treatment method herein, and use of an anti-TGFβ antibody or an antigen-binding fragment thereof in the manufacture of a medicament for treating osteogenesis imperfecta in the treatment method herein.

Also provided is an article of manufacture (e.g., a kit), comprising an anti-TGFβ antibody or an antigen-binding fragment thereof for use in treating osteogenesis imperfecta in the treatment method herein.

Other features, objects, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE FIGURES

FIG. 1 shows human bone specimen processing for histology, RNA, and protein studies. Panel A: an illustration of bone specimen pulverization environment showing that bone specimen was immersed in double-layered liquid nitrogen and pulverized by electric-powered drill. Panel B: a representation of a bone specimen prior to processing is shown at the upper right corner. After pulverization, bone powder at the bottom of a pestle was collected in liquid nitrogen. A 2-3 mm³ area immediately next to the pulverized region was processed for histology and immunochemistry.

FIG. 2 show bone histology of individuals with OI type III and unaffected controls. Panel A: whole images of H&E stain histological sections of collected bone specimens. The majority of specimens from unaffected individuals were cortical bone. The OI type III specimens were more heterogeneous. Whereas the majority were cortical bone, two specimens (OI62 and OI83) contained only trabecular bone. One specimen (OI41) had fibrous cartilage and one specimen (OI35) contained callus and trabecular bone. Scale bar: 200 μm. Panel B: representative higher magnification (10×) H&E stain images of unaffected control and OI type III bones. In the control bone, the well-organized Haversian canal system was observed while the OI type III bone showed a more woven cortical bone with less organized Haversian canal and visible more osteocytes with a more sphere shape. Scale bar: 50 μm.

FIG. 3 shows an increased osteocyte density in type III OI bone. Quantification of osteocyte density in unaffected control (n=4) and OI type III (n=10). Each dot represents osteocyte density of one individual. Mean and standard deviation are depicted.

FIG. 4 shows expression changes in genes using Nanostring and RNA Sequence platforms. 1 non-OI control and 1 type III OI with both RNA-seq and NanoString data were included in the analysis. 155 genes that fulfill NanoString quality controls were used for the validation of RNA-seq differential expression data. Fold change direction (increased or decreased compare to non-OI control) from RNA-seq and NanoString of the 155 genes is presented as red up-arrow (increased in type III OI) or blue down-arrow (decreased in type III OI). Purple background: inconsistent fold change direction between the two platform. White backgrounds: consistent fold change direction between the two platforms. The consistent rate is 92%.

FIG. 5 represents a transcriptomic and bioinformatics analysis demonstrating activation of TGFβ signaling in type III OI bone. Panel A shows a principle component analysis (PCA) plot of all the control and OI type III in 3-PC dimensions. Panel B shows hierarchical clustering based on Euclidian distance using RPKM of all control and OI type III bone data. Blue: down-regulated; yellow: up-regulated. Panel C shows a gene set enrichment plot demonstrated activation of TGFβ signaling. NES: normalized enrichment score. FDR: false discovery rate. The expression pattern of genes involved in the TGFβ gene set in the analysis database. Blue: down-regulated. Red: up-regulated. C: control. OI: OI type III.

FIG. 6 shows a significantly enriched Gene Ontology (GO) analysis results with enrichment fold-change in Type III OI bone. Partek GO enrichment results under the categories of skeletal processes, bone cells, collagen, and skeletal-related major signaling pathways are provided. Significance was defined by P-value<0.05.

FIG. 7 shows the top 20 gene set enrichment assay (GSEA) results of type III OI and control transcription profiles. The top 20 enriched gene set in type III OI or in non-OI control are provided. NES: Normalized Enrichment Score; FDR q-val: false discovery rate adjusted q-value.

FIG. 8 shows significantly changed proteins in RPPA analysis. The table shows the complete list of significantly changed proteins based on nominal P-value<0.05 in Type III OI bone. The protein expression levels are presented as normalized intensity in the protein array.

FIG. 9 shows an increase of phosphorylated SMAD2 (pSMAD2) level in OI type III bone. Panel A shows immunohistochemistry staining of pSMAD2 in control and OI type III bone sections. Higher magnification images are shown at lower right black boxes. Increase pSMAD2 signal was detected in all OI samples, especially in osteocytes. Scale bar: 20 μm. Panel B shows a western blot of phosphorylated SMAD2(p-SMAD2) and total SMAD2 (T-SMAD2) in protein extracted from control and OI type III bone. 50 μg of total protein was loaded. An additional OI62 sample was treated with calf-intestinal alkaline phosphatase (CIP) to remove phosphorylation signal and served as a negative control for accurate pSMAD2 signal (indicated by arrow). Panel C shows quantification result of western blot in (B) showing as ratio of phosphorylated (phospho) versus total SMAD2. GAPDH was used as loading control. C: control; OI: OI type III.

FIG. 10 shows hematological safety data from the trial evaluating safety of fresolimumab in adults with OI. A drop in hemoglobin was noted in two participants (FR005 and FR009), both graded as mild. These two individuals had epistaxis that was graded as being related to the study medication and menstrual bleeding that was not graded as being unrelated to study medication. The platelet count and the INR were within normal limits.

FIG. 11 illustrates the effect of fresolimumab on bone turnover markers and bone density. An increase in osteocalcin (Ocn) and C-terminal telopeptide (CTX) levels were observed in three out of the four participants in the 1 mg·kg body weight-1 cohort with the peak values being observed between days 30 and 90 after treatment. In the 4 mg·kg body weight-1 cohort, a decrease in Ocn was observed on day 30 and this suppression persisted through day 180. In participant, FR012, obtain bone turnover markers on day 30 could not be obtained due to an inability to travel. Two individuals with OI type IV had robust increase in L1-4 aBMD in both dose cohorts.

FIG. 12 illustrates the correlation and agreement between the two central reads for LS aBMD. The LS aBMD was read by two independent readers who were blinded to the trial design. There was a strong correlation between the two reads (R=0.995). A Bland-Altman plot showed a high degree of agreement between the two reads. The mean difference, upper limit of agreement, lower limit of agreement, and the confidence intervals are depicted in blue, green, and red, respectively.

### DETAILED DESCRIPTION OF THE INVENTION

The present disclosure provides a method of treating moderate-to-severe OI (e.g., type IV OI) in a human patient by administering a monoclonal antibody that binds and neutralizes all isoforms of human TGFβ. The method is based on the surprising discovery that infrequent dosing (e.g., every three or six months) of an anti-TGFβ antibody may be sufficient to improve the symptoms of OI in patients.

Standard of care therapy for OI bone fragility involves repurposing of medications that are used to treat osteoporosis. However, given the heterogeneity of OI and the variability in the clinical study designs, the effects of BPN have been inconsistent. Further, in a randomized trial involving adults with OI, treatment with an anabolic agent, teriparatide, led to increase in aBMD and vBMD in individuals with the mild form (OI type I) but not in the moderate-to-severe forms of the disorder (OI types III and IV). None of these repurposed therapies addresses specific pathogenetic mechanism in OI, and hence, they have no effect on extraskeletal manifestations. The present inventors have surprisingly found that individuals with moderate-to-severe OI who were treated with a 1 or 4 mg/kg single dose of an anti-TFGβ antibody showed a robust increase in lumbar spine areal bone mineral density (LS aBMD).

Targeting TGFβ signaling in bone offers significant pharmacodynamic advantages. While modulating such a pivotal pathway in extraskeletal tissues requires sustained pharmacological inhibition, the human bone remodeling unit is approximately 3 months. Thus, pharmacological inhibition at a single-time-point may have prolonged effects beyond the terminal half-life and persistence of the drug in circulation. It has been shown herein that treatment with even a single dose a pan-specific anti-TGFβ antibody was associated with changes in bone turnover and aBMD at day 90 and 180. In addition, the low dosing frequency offers safety advantages due to lower cumulative dosage, allowing for reduction of systemic toxicity. The efficacy of low-frequency dosing is surprising because in preclinical studies in OI mice, treatment with an anti-TGFβ antibody (murine antibody 1D11) at a frequency of 3 times a week led to findings of improvement but the improvement diminished when the mice were dosed at less frequency (e.g., at Q4W).

### I. Osteogenesis Imperfecta

OI encompasses a group of congenital bone disorders characterized by deficiencies in one or more proteins involved in bone matrix deposition or homeostasis. There are over 19 types of OI that are defined by their specific gene mutation, the resulting protein deficiency, and phenotype of the affected individual. The classification includes findings on X-rays and other imaging tests. The main OI types are as follows (information from a website of The John Hopkins University).

Type I is the mildest and most common type. About 50% of all affected children have this type. There are few fractures and deformities

Type II is the most severe type. A baby has very short arms and legs, a small chest, and soft skull. He or she may be born with fractured bones and may also have a low birth weight and lungs that are not well developed. A baby with type II OI usually dies within weeks of birth

Type III is the most severe type in babies who don't die as newborns. At birth, a baby may have slightly shorter arms and legs than normal and arm, leg, and rib fractures. A baby may also have a larger than normal head, a triangle-shaped face, a deformed chest and spine, and breathing and swallowing problems.

Type IV is an OI type where symptoms are between mild and severe. A baby with type IV may be diagnosed at birth. He or she may not have any fractures until crawling or walking. The bones of the arms and legs may not be straight. He or she may not grow normally.

Type V is similar to type IV. Symptoms may be medium to severe. It is common to have enlarged thickened areas (hypertrophic calluses) in the areas where large bones are fractured.

Type VI is very rare. Symptoms are medium and similar to type IV.

Type VII may be like type IV or type II. It is common to have shorter than normal height. It is also common to have shorter than normal upper arm and thighbones.

Type VIII is similar to types II and III. The patient has very soft bones and severe growth problems.

Although phenotypes vary among OI types, common symptoms include incomplete ossification of bones and teeth, reduced bone mass, brittle bones, and pathologic fractures. Specific symptoms include easily broken bones, bone deformities (such as bowing of the legs), discoloration of the white of the eye (sclera), a barrel-shaped chest, a curved spine, a triangle-shaped face, loose joints, muscle weakness, skin that easily bruises, hearing loss in early adulthood, and/or soft, discolored teeth. Complications of OI include respiratory infections (e.g., pneumonia), heart problems (e.g., poor heart valve function), kidney stones, joint problems, hearing loss, and abnormal eye conditions (including vision loss). OI may be diagnosed or monitored by X-rays, lab tests (e.g., blood test and genetic testing), dual energy X-ray absorptiometry scan (DXA or DEXA scan), and bone biopsy.

While multiple pathogenic genetic mutations can cause the various subtypes of OI, more than 90% are caused by pathogenic variants in the COL1A1 gene (which encodes collagen type I alpha 1 chain) or the COL2A1 gene (which encodes collagen type II alpha 1 chain), or genes encoding proteins that post-translationally modify type I collagen (CRTAP, PPIB and LEPRE1) (Patel et al., ibid; Lim et al., Bone (2017)102:40-49).

The treatment method of the present disclosure is effective in treating moderate-to-severe forms of OI, such as type IV OI. In some embodiments, the OI in the patient is caused by a mutation (e.g., a glycine substitution) in COL1A1 or COL1A2 or by biallelic pathogenic variants in CRTAP, PPIB, or LEPRE1. See, e.g., mutations shown in Tables 1 and 3 below.

### II. Anti-TGFβ Antibodies

TGFβs are multifunctional cytokines that are involved in cell proliferation and differentiation, embryonic development, extracellular matrix formation, bone development, wound healing, hematopoiesis, and immune and inflammatory responses. Secreted TGFβ protein is cleaved into a latency-associated peptide (LAP) and a mature TGFβ peptide, and is found in latent and active forms. The mature TGFβ peptide forms both homodimers and heterodimers with other TGFβ family members.

There are three human (h) TGFβ isoforms: TGFβ1, TGFβ2, and TGFβ3 (UniProt Accession Nos. P01137, P08112, and P10600, respectively). TGFβ1 differs from TGFβ2 by 27, and from TGFβ3 by 22, mainly conservative, amino acids. Human TGFβs are very similar to mouse TGFβs: human TGFβ1 has only one amino acid difference from mouse TGFβ1; human TGFβ2 has only three amino acid differences from mouse TGFβ2; and human TGFβ3 is identical to mouse TGFβ3.

Binding of a TGFβ protein to a homodimeric or heterodimeric TGFβ transmembrane receptor complex activates the canonical TGFβ signaling pathway mediated by intracellular SMAD proteins. Deregulation of TGFβs leads to pathological processes that, in humans, have been implicated in numerous conditions, such as birth defects, cancer, chronic inflammatory, autoimmune diseases, and fibrotic diseases (see, e.g., Border et al., Curr Opin Nephrol Hypertens. (1994) 3(4):446-52; Border et al., Kidney Int Suppl. (1995) 49:S59-61).

For the present OI treatment methods, the anti-TGFβ antibody may be a pan-specific antibody, i.e., an antibody that binds and neutralizes all three isoforms of TGFβ with high affinity. In some embodiments, the antibody is fresolimumab. Fresolimumab is a recombinant human antibody. Its heavy chain is shown below:

(SEQ ID NO: 1)

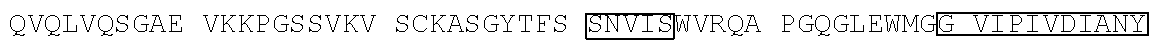

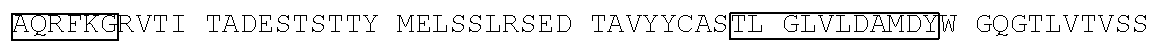

ASTKGPSVFP LAPCSRSTSE STAALGCLVK DYFPEPVTVS WNSGALTSGV HTFPAVLQSS
GLYSLSSVVT VPSSSLGTKT YTCNVDHKPS NTKVDKRVES KYGPPCPSCP APEFLGGPSV
FLFPPKPKDT LMISRTPEVT CVVVDVSQED PEVQFNWYVD GVEVHNAKTK PREEQENSTY
RVVSVLTVLH QDWLNGKEYK CKVSNKGLPS SIEKTISKAK GQPREPQVYT LPPSQEEMTK
NQVSLTCLVK GFYPSDIAVE WESNGQPENN YKTTPPVLDS DGSFFLYSRL TVDKSRWQEG
NVFSCSVMHE ALHNHYTQKS LSLSLGK

In the above sequence, positions 1-120 is the heavy chain variable domain (V_{H}), and the heavy chain CDRs (“HCDRs”; according to Kabat definition) are boxed. This heavy chain comprises a human IgG₄ constant region.

The light chain of fresolimumab is shown below:

(SEQ ID NO: 2)
ETVLTQSPGT LSLSPGERAT LSCRASQSLG SSYLAWYQQK PGQAPRLLIY GASSRAPGIP
DRFSGSGSGT DFTLTISRLE PEDFAVYYCQ QYADSPITFG QGTRLEIKRT VAAPSVFIFP
PSDEQLKSGT ASVVCLLNNF YPREAKVQWK VDNALQSGNS QESVTEQDSK DSTYSLSSTL
TLSKADYEKH KVYACEVTHQ GLSSPVTKSF NRGEC

In the above sequence, positions 1-108 is the light chain variable domain (V_{L}), and the light chain CDRs (“LCDRs”; according to Kabat definition) are underlined. This light chain comprises a human Cκ constant region.

In some embodiments, the anti-TGFβ antibody herein is Ab1, a variant of fresolimumab. The heavy chain of Ab1 differs from that of fresolimumab in only a residue in the IgG₄ hinge region. The residue is S228 (Eu numbering), where Ab1 has a proline at that position, i.e., having a S228P substitution relative to fresolimumab. Ab1 and fresolimumab have the same light chain. The heavy chain of Ab1 is shown below:

(SEQ ID NO: 3)

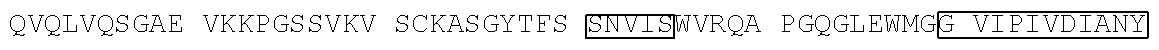

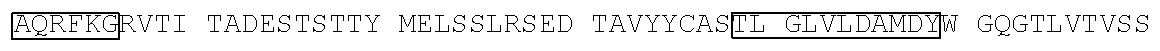

ASTKGPSVFP LAPCSRSTSE STAALGCLVK DYFPEPVTVS WNSGALTSGV HTFPAVLQSS

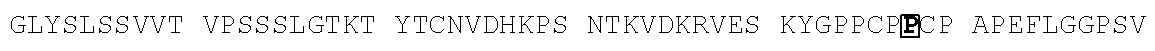

FLFPPKPKDT LMISRTPEVT CVVVDVSQED PEVQFNWYVD GVEVHNAKTK PREEQFNSTY
RVVSVLTVLH QDWLNGKEYK CKVSNKGLPS SIEKTISKAK GQPREPQVYT LPPSQEEMTK
NQVSLTCLVK GFYPSDIAVE WESNGQPENN YKTTPPVLDS DGSFFLYSRL TVDKSRWQEG
NVFSCSVMHE ALHNHYTQKS LSLSLGK

In the above sequence, the HCDRs are boxed, and the S228P substitution is boxed and boldfaced.

In some embodiments, the anti-TGFβ antibody comprises one or more (e.g., all six) of the HCDR1-3 and the LCDR1-3 of fresolimumab. In other words, the antibody comprises one or more (e.g., all six) of the following HCDRs and LCDRs:

HCDR1
(SEQ ID NO: 4)
SNVIS
HCDR2
(SEQ ID NO: 5)
GVIPIVDIANYAQRFKG
HCDR3
(SEQ ID NO: 6)
TLGLVLDAMDY
LCDR1
(SEQ ID NO: 7)
RASQSLGSSYLA
LCDR2
(SEQ ID NO: 8)
GASSRAP
LCDR3
(SEQ ID NO: 9)
QQYADSPIT

In some embodiments, the anti-TGFβ antibody comprises the V_{H} and/or V_{L} of fresolimumab or Ab1. In other words, the antibody comprises one or both of the following sequences:

V_{H}:
(SEQ ID NO: 10)
QVQLVQSGAE VKKPGSSVKV SCKASGYTFS SNVISWVRQA PGQGLEWMGG VIPIVDIANY
AQRFKGRVTI TADESTSTTY MELSSLRSED TAVYYCASTL GLVLDAMDYW GQGTLVTVSS
V_{L}:
(SEQ ID NO: 11)
ETVLTQSPGT LSLSPGERAT LSCRASQSLG SSYLAWYQQK PGQAPRLLIY GASSRAPGIP
DRFSGSGSGT DFTLTISRLE PEDFAVYYCQ QYADSPITFG QGTRLEIK

In some embodiments, the anti-TGFβ antibody is of a human IgG isotype, such as human IgG₄ isotype. In certain embodiments, the human IgG₄ constant region comprises the following amino acid sequence:

(SEQ ID NO: 12)
ASTKGPSVFP LAPCSRSTSE STAALGCLVK DYFPEPVTVS WNSGALTSGV HTFPAVLQSS

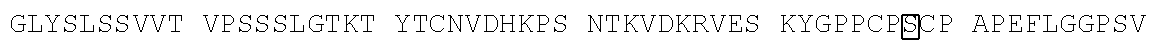

FLFPPKPKDT LMISRTPEVT CVVVDVSQED PEVQFNWYVD GVEVHNAKTK PREEQENSTY
RVVSVLTVLH QDWLNGKEYK CKVSNKGLPS SIEKTISKAK GQPREPQVYT LPPSQEEMTK
NQVSLTCLVK GFYPSDIAVE WESNGQPENN YKTTPPVLDS DGSFFLYSRL TVDKSRWQEG
NVFSCSVMHE ALHNHYTQKS LSLSLGK

In further embodiments, the human IgG₄ constant region has a mutation at position 228 (Eu numbering). In some embodiments (e.g., Ab1), the mutation is a serine-to-proline mutation (S228P). In the above sequence, the S228 serine is boxed.

In some embodiments, the anti-TGFβ antibody (e.g., Ab1 and fresolimumab) comprises a human κ light chain constant region (Cκ). In certain embodiments, the human Cκ comprises the amino acid sequence:

(SEQ ID NO: 13)
RTVAAPSVFI FPPSDEQLKS GTASVVCLLN NFYPREAKVQ WKVDNALQSG NSQESVTEQD
SKDSTYSLSS TLTLSKADYE KHKVYACEVT HQGLSSPVTK SFNRGEC

In some embodiments, an antigen-binding fragment of a full anti-TGFβ antibody may also be used. The term “antigen-binding fragment” or a similar term refers to the portion of an antibody that comprises the amino acid residues that interact with an antigen and confer on the binding agent its specificity and affinity for the antigen. Non-limiting examples of antigen-binding fragments include: Fab fragments, F(ab′)₂ fragments, Fd fragments, Fv fragments, single chain Fv (scFv), dAb fragments, and minimal recognition units consisting of the amino acid residues that mimic the hypervariable domain of the antibody.

In some embodiments, the antibody or antigen-binding fragment herein is connected to the bone-targeting moiety. In further embodiments, the bone-targeting moiety is a poly-arginine (poly-D) peptides. As used herein, the term “poly-D peptide” refers to a peptide sequence having a plurality of aspartic acid or aspartate or “D” amino acids, such as about 2, 3, 4, 5, 6, 7, 8, 9, 10, 20, 30, or more aspartic acid amino acids (residues). For example, a poly-D peptide can include about 2 to about 30, or about 3 to about 15, or about 4 to about 12, or about 5 to about 10, or about 6 to about 8, or about 7 to about 9, or about 8 to about 10, or about 9 to about 11, or about 12 to about 14 aspartic acid residues. Poly-D peptides may include only aspartate residues, or may include one or more other amino acids or similar compounds. As used herein, the term “D10” refers to a contiguous sequence of ten aspartic acid amino acids, as seen in SEQ ID NO:14. In some embodiments, an antibody or antibody fragment of the invention may include 1, 2, 3, 4, 5, 6, 7, 8, 9, 10, 11, 12, or more than 12 poly-D peptides.

The poly-D peptide can be connected to the anti-TGFβ antibody or antigen-binding fragment by fusion via recombinant technology, such that the poly-D is connected to the antibody or fragment through a peptidyl bond (i.e., the antibody or fragment is a fusion protein). For example, a poly-D peptide can be fused to the N- or C-terminus, or both, of the heavy chain, and/or the N- or C-terminus, or both, of the light chain. The poly-D peptide also can be connected to the anti-TGFβ antibody or antigen-binding fragment by chemical conjugation, e.g., by chemical reaction with a cysteine or lysine residue on the antibody or antibody-binding fragment with or without a linker moiety (e.g., a maleimide function group and a polyethylene glycol (PEG)). See, e.g., WO 2018/136698.

In certain embodiments, the antibody is fresolimumab fused to a D10 peptide at the N-terminus, C-terminus, or both termini, of the heavy chain. In some embodiments, the antibody is fresolimumab fused to a D10 peptide at the C-terminus of the light chain. In particular embodiments, the antibody is fresolimumab fused to a D10 peptide at both termini of the heavy chain and at the C-terminus of the light chain.

In certain embodiments, the antibody is Ab1 fused to a D10 peptide at the N-terminus, C-terminus, or both termini, of the heavy chain. In some embodiments, the antibody is Ab1 fused to a D10 peptide at the C-terminus of the light chain. In particular embodiments, the antibody is Ab1 fused to a D10 peptide at both termini of the heavy chain and at the C-terminus of the light chain.

The anti-TGFβ antibody or antigen-binding fragment thereof of the present disclosure can be made by methods well established in the art. DNA sequences encoding the heavy and light chains of the antibodies can be inserted into expression vectors such that the genes are operatively linked to necessary expression control sequences such as transcriptional and translational control sequences. Expression vectors include plasmids, retroviruses, adenoviruses, adeno-associated viruses (AAV), plant viruses such as cauliflower mosaic virus, tobacco mosaic virus, cosmids, YACs, EBV derived episomes, and the like. The antibody light chain coding sequence and the antibody heavy chain coding sequence can be inserted into separate vectors, and may be operatively linked to the same or different expression control sequences (e.g., promoters). The expression vectors encoding the antibodies of the present disclosure are introduced to host cells for expression. The host cells are cultured under conditions suitable for expression of the antibody, which is then harvested and isolated. Host cells include mammalian, plant, bacterial or yeast host cell. Mammalian cell lines available as hosts for expression are well known in the art and include many immortalized cell lines available from the American Type Culture Collection (ATCC). These include, inter alia, Chinese hamster ovary (CHO) cells, NSO cells, SP2 cells, HEK-293T cells, 293 Freestyle cells (Invitrogen), NIH-3T3 cells, HeLa cells, baby hamster kidney (BHK) cells, African green monkey kidney cells (COS), human hepatocellular carcinoma cells (e.g., Hep G2), A549 cells, and a number of other cell lines. Cell lines may be selected based on their expression levels. Other cell lines that may be used are insect cell lines, such as Sf9 or Sf21 cells. Tissue culture media for the host cells may include, or be free of, animal-derived components (ADC), such as bovine serum albumin. In some embodiments, ADC-free culture media is preferred for human safety. Tissue culture can be performed using the fed-batch method, a continuous perfusion method, or any other method appropriate for the host cells and the desired yield.

### III. Pharmaceutical Compositions and Use

The methods described herein comprise administering a therapeutically effective amount of an anti-TGFβ antibody or antigen-binding fragment thereof to an OI patient. As used herein, the phrase “therapeutically effective amount” means a dose of antibody that binds to TGFβ that results in a detectable improvement in one or more symptoms associated with moderate-to-severe OI (e.g., type IV OI) or which causes a biological effect (e.g., a decrease in the level of a particular biomarker) that is correlated with the underlying pathologic mechanism(s) giving rise to the condition or symptom(s) of moderate-to-severe OI.

Improvement of OI can be manifested in decreased bone turnover, reduced rates of bone remodeling, and/or decreased osteocyte density. In some embodiments, improvement in OI is indicated by improvement of a bone parameter selected from the group consisting of bone mineral density (BMD), bone volume density (BV/TV), total bone surface (BS), bone surface density (BS/BV), trabecular number (Tb.N), trabecular thickness (Tb.Th), trabecular spacing (Tb.Sp), and total volume (Dens TV).

In certain embodiments, the improved bone parameter is lumbar spine areal BMD (LS aBMD), as determined by dual-energy X-ray absorptiometry. Compared to baseline level prior to treatment, the LS aBMD value may increases by at least 1%, e.g., by at least 1, 2, 3, 4, 5, 6, 7, 8, 9, 10, 150, 20, or more percent.

In some embodiments, BMD, bone mass, and/or bone strength are increased by about 5% to about 200% following treatment with a therapeutically effective amount of the anti-TGFβ antibody or fragment. In certain embodiments, BMD, bone mass, and/or bone strength are increased by about 5% to about 10%, 10% to about 15%, 15% to about 20%, 20% to about 25%, 25% to about 30%, 30% to about 35%, 35% to about 40%, 40% to about 45%, 45% to about 50%, 50% to about 55%, 55% to about 60%, 60% to about 65%, 65% to about 70%, 70% to about 75%, 75% to about 80%, 80% to about 85%, 85% to about 90%, 90% to about 95%, 95% to about 100%, 100% to about 105%, 105% to about 110%, 110% to about 115%, 115% to about 120%, 120% to about 125%, 125% to about 130%, 130% to about 135%, 135% to about 140%, 140% to about 145%, 145% to about 150%, 150% to about 155%, 155% to about 160%, 160% to about 165%, 165% to about 170%, 170% to about 175%, 175% to about 180%, 180% to about 185%, 185% to about 190%, 190% to about 195%, or 195% to about 200%, following treatment.

In some embodiments, the therapeutically effective amount may lead to decreased bone turnover, e.g., as indicated by a decrease in serum or urinary biomarker such as urinary hydroxyproline, urinary total pyridinoline (PYD), urinary free deoxypyridinoline (DPD), urinary collagen type-I cross-linked N-telopeptide (NTX), urinary or serum collagen type-I cross-linked C-terminal telopeptide (CTX), bone sialoprotein (BSP), osteopontin (OPN), and tartrate-resistant acid phosphatase 5b (TRAP). In certain embodiments, the decrease, as compared to baseline level (e.g., before treatment), is by about 5% to about 200% following treatment with an antibody that binds to TGFβ. For example, the decrease may be about 5% to about 10%, 10% to about 15%, 15% to about 20%, 20% to about 25%, 25% to about 30%, 30% to about 35%, 35% to about 40%, 40% to about 45%, 45% to about 50%, 50% to about 55%, 55% to about 60%, 60% to about 65%, 65% to about 70%, 70% to about 75%, 75% to about 80%, 80% to about 85%, 85% to about 90%, 90% to about 95%, 95% to about 100%, 100% to about 105%, 105% to about 110%, 110% to about 115%, 115% to about 120%, 120% to about 125%, 125% to about 130%, 130% to about 135%, 135% to about 140%, 140% to about 145%, 145% to about 150%, 150% to about 155%, 155% to about 160%, 160% to about 165%, 165% to about 170%, 170% to about 175%, 175% to about 180%, 180% to about 185%, 185% to about 190%, 190% to about 195%, or 195% to about 200%, following treatment.

In some embodiments, the therapeutically effective amount may lead to an increase in the level of serum or urine biomarker of bone deposition, such as total alkaline phosphatase, bone-specific alkaline phosphatase, osteocalcin (OCN), and type-I procollagen (C-terminal/N-terminal). In certain embodiments, the increase, as compared to the baseline level (e.g., prior to treatment), is by about 5% to about 200% following treatment. For example, the increase may be by about 5% to about 10%, 10% to about 15%, 15% to about 20%, 20% to about 25%, 25% to about 30%, 30% to about 35%, 35% to about 40%, 40% to about 45%, 45% to about 50%, 50% to about 55%, 55% to about 60%, 60% to about 65%, 65% to about 70%, 70% to about 75%, 75% to about 80%, 80% to about 85%, 85% to about 90%, 90% to about 95%, 95% to about 100%, 100% to about 105%, 105% to about 110%, 110% to about 115%, 115% to about 120%, 120% to about 125%, 125% to about 130%, 130% to about 135%, 135% to about 140%, 140% to about 145%, 145% to about 150%, 150% to about 155%, 155% to about 160%, 160% to about 165%, 165% to about 170%, 170% to about 175%, 175% to about 180%, 180% to about 185%, 185% to about 190%, 190% to about 195%, or 195% to about 200%, following treatment.

In some embodiments, the therapeutically effective amount promotes bone deposition. In some embodiments, the therapeutically effective amount improves the function of a non-skeletal organ affected by OI, such as hearing, vision, lung function, and kidney function.

The therapeutically effective amount may be 1-10 mg/kg, e.g., 1, 2, 3, 4, 5, 6, 7, 8, 9, or 10 mg/kg. In some embodiments, the OI patient is treated with this amount of fresolimumab or Ab1 by intravenous injection. The treatment may be repeated at an interval as deemed appropriate by a physician for the patient. In some embodiments, the treatment with the anti-TGFβ antibody or antigen-binding fragment thereof may be repeated every month, every two months, every three months, every four months, every five months, every six months, every nine months, every 12 months, or every 18 months.

The patients may be adults (e.g., patients 18 years or older). The patients may be pediatric patients (patients who are younger than 18 years old, e.g., patients who are newborn to 6 years old, who are 6 to 12 years old, or who are 12 to 18 years old).

### IV. Combination Therapies

In some embodiments, the present anti-TGFβ antibody therapy may be combined with other OI treatment. Examples of additional therapeutic agents include, but are not limited to, bisphosphonates, calcitonin, teriparatide, and any other compound known to treat, prevent, or ameliorate OI. The additional therapeutic agent(s) can be administered concurrently or sequentially with the antibody that binds to TGFβ. Examples of bisphosphonates are etidronate, clodronate, tiludronate, pamidronate, neridronate, olpadronate, alendronate, ibandronate, zoledronate, and risedronate. In some embodiments, the additional therapeutic agent is a drug that stimulates bone formation such as parathyroid hormone analogs and calcitonin.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, cell and tissue culture, molecular biology, immunology, and protein and nucleic acid chemistry and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art.

In order that this invention may be better understood, the following examples are set forth. These examples are for purposes of illustration only and are not to be construed as limiting the scope of the invention in any manner.

### EXAMPLES

In the following Examples, histology and RNA-seq were performed on bones obtained from children affected (n=10) and unaffected (n=4) by OI. Gene Ontology (GO) enrichment assay, gene set enrichment assay (GSEA), and Ingenuity Pathway Analysis (IPA) were used to identify key dysregulated pathways and regulators. Reverse-phase protein array (RPPA), Western blot (WB), and Immunohistochemistry (IHC) were performed to confirm the changes at protein level. A phase 1 study with a single administration of 1 or 4-mg/kg dose of fresolimumab, a pan-anti-TGFβ neutralizing antibody, was conducted in eight adults with OI types III and IV. Safety of fresolimumab and its effects on lumbar spine areal bone mineral density (LS aBMD) and bone remodeling markers were assessed. Details of the materials and methods for the study are as follows.

### Human Bone Samples Collection and Processing

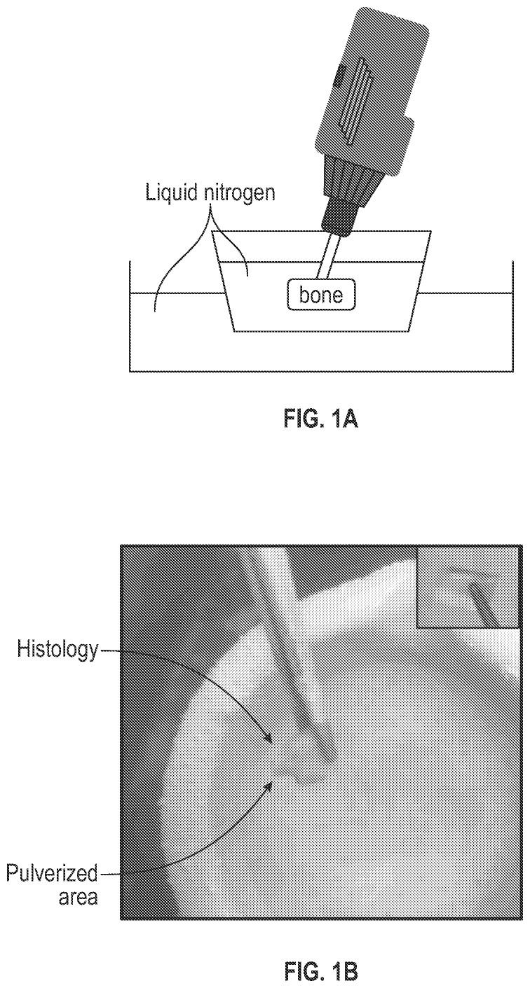

Bones from children with OI and children unaffected with OI were obtained under a protocol approved by the Institutional Review Board (IRB) of Baylor College of Medicine (BCM), Houston, TX, USA. Bone samples were obtained from children who were already undergoing surgery for a medical reason. Fragments of bone removed during surgery which otherwise would have been discarded, were collected and processed. Informed consent was obtained from the parents or legal guardians prior to collection of all samples. Bone specimens were processed in a liquid nitrogen-based environment as described in FIG. 1 following a previously reported protocol (Chou et al., Osteoarthritis Cartilage (2013) 21(3):450-61).

### RNA Extraction, RNA-Seq, Validation and Data Analysis

Total RNA from pulverized bone was extracted using TRIzol® (ThermoFisher Scientific) and further purified by lithium chloride precipitation. RNA quality and quantity were measured by Bioanalyzer (Agilent Technologies, Santa Clara, CA, USA). Total RNA was then subjected to RNA-seq followed by validation and bioinformatic analyses for pathways and upstream regulators.

### Protein Extraction, Reverse Phase Protein Array (RPPA), Western Blotting (WB), and Immunohistochemistry (IHC)

5 Protein was extracted from pulverized bone by using lysis buffer overnight in 4° C. (250 mM EDTA, 6M guanidine-HCl, 50 mM Tris-HCl, pH 7.4). The extract was concentrated by methanol-water-chloroform precipitation and dissolved in 4% SDS buffer (4SB; 4% SDS, 50 mM Tris, 5 mM EDTA, pH 7.4) for WB or diluted into 0.5 mg/ml in SDS sample buffer for RPPA. In total, 3 controls and 5 OI type III bone samples were included in WB; 4 controls and 8 type III OI bone samples were included in the RPPA and IHC (Table 1).

TABLE 1

| | | | | | |
|---|---|---|---|---|---|
| | | | Anatomic | | |
| ID | Age | Diagnosis | location | Mutation | Experiment performed |

Non-OI Control

| | | | | | |
|---|---|---|---|---|---|
| C5 | 12 | acetabular displasia | Femur | NA | HE, osteocyte density, IHC, WB, |
| | | | | | RPPA |
| C14 | 10 | bilateral congenital | Femur | NA | HE, osteocyte density, IHC, WB, |
| | | subluxation of hip | | | RNA-Seq, RPPA |
| C15 | 11 | cerebral palsy, | Femur | NA | HE, osteocyte density, IHC, WB, |
| | | hip dislocation | | | RNA-Seq, RPPA |
| C18 | 10 | cerebral palsy, acquired | Femur | NA | HE, osteocyte density, IHC, RNA- |
| | | subluxation of hip | | | Seq, NanoString ®, RPPA |

OI Type III

| | | | | | |
|---|---|---|---|---|---|
| OI62 | 6 | OI type III | Femur or | COL1A1 | HE, osteocyte density, IHC, WB, |
| | | | Tibia | p.Gly866Ser | RPPA |
| OI33 | 8 | OI type III | Femur or | COL1A2 | HE, osteocyte density, IHC, WB, |
| | | | Tibia | p.Gly760Arg | RNA-Seq, RPPA |
| OI41 | 4 | OI type III | Femur | COL1A1 | HE, IHC, RPPA |
| | | | | p.Gly821Ser | |
| OI91 | 1.8 | OI type III | Tibia | COL1A1 | HE, osteocyte density, IHC, RPPA |
| | | | | p.Gly767Ser | |
| OI83 | 4 | OI type III | Femur | COL1A2 | HE, osteocyte density, IHC, RPPA |
| | | | | p.Gly949Ser | |
| OI35 | 9 | OI type III | Tibia | COL1A2 | HE, IHC,WB |
| | | | | p.Gly358Ser | |
| OI3 | 2 | OI type III | Femur or | COL1A1 | HE, osteocyte density, IHC, RPPA |
| | | | Tibia | | |
| OI13 | 3 | OI type III | Tibia | COL1A1 | HE, osteocyte density, IHC, RNA- |
| | | | | p.Gly821Ser | Seq, RPPA |
| OI85 | 8 | OI type III | Tibia | COL1A2 | HE, osteocyte density, WB, RNA- |
| | | | | p.del345Val | Seq, NanoString ®, RPPA |
| OI70 | 3 | OI type III | Femur | COL1A1 | HE, osteocyte density, WB |
| | | | | p.Gly857Cys | |

### Phase 1 Clinical Study Design

A phase 1, dose-escalating, clinical trial evaluating fresolimumab in adults with moderate-to severe forms of OI was conducted as a part of the National Institutes of Health Rare Disease Clinical Research Network's Brittle Bone Disorders Consortium. Stage 1 of the study involved a single infusion of fresolimumab (1 mg/kg body weight and 4 mg/kg body weight; n=4 in each dose cohort). The total follow-up period was 6 months. The primary outcome measure was safety of single dose fresolimumab. The secondary outcomes were to assess the effects of fresolimumab on lumbar spine areal bone mineral density (LS aBMD) by dual-energy X-ray absorptiometry (DXA) and bone turnover markers (Ocn and CTX) in blood.

Individuals over 18 years of age with a diagnosis of moderate-to-severe OI based on having sustained 20 or more fractures and a glycine substitution mutation in COL1A1 or COL1A2 or biallelic pathogenic variants in CRTAP, PPIB, or LEPRE1 were enrolled.

Exclusion criteria were: 1) instrumentation at LS and both hips precluding assessment of aBMD, 2) long bone fractures three months prior to screening, 3) treatment with oral BPN within 6 months of screening or with intravenous BPN and teriparatide within 12 months of screening, 4) expected skeletal surgery, 5) having characteristics that could affect safety such as autoimmune disease, tuberculosis, history of cancer or precancerous lesions, cardiac valvular disease, and bleeding diathesis. Markers of bone turnover were measured by CLIA- and CAP-certified laboratories. DXA scan was performed using validated clinical machines at Texas Children's Hospital. The scans were read by two central readers who were blinded to study procedures.

### Histology and Osteocyte Density Analysis

The processed bone specimens were fixed in 4% paraformaldehyde (Sigma-Aldrich, St. Louis, MO, USA) for 48 hours and decalcified in 10% EDTA (Sigma-Aldrich) for 14 days in 4° C. Paraffin sections were stained with hematoxylin and eosin for morphological and osteocyte number analysis. Osteocyte density was calculated using BIOQUANT OSTEO (BIOQUANT Image Analysis Corporation, Nashville, TN, USA).

### RNA-Seq and Data Analysis

For RNA-Seq, 250 ng of total RNA was used for TureSeq Stranded mRNA library preparation (Illumina, San Diego, CA, UAS) with ERCC spike-in (ThermoFisher Scientific) applied according to manufacturer's instructions. Twenty-two pM of equimolarly pooled library was loaded onto one lane of a high output v4 flow cell for bridge amplification using the Illumina cBot machine. A paired-end 100 cycle run was used to sequence the flow cell on a HiSeq 2500 Sequencing System in High Output Mode with v4 chemistry (FC-401-4003, Illumina). PhiX Control v3 adapter-ligated library (Illumina) was spiked-in at 2% by weight to ensure balanced diversity and to monitor clustering and sequencing performance. An average of 42.5 million paired-end reads was generated for each sample. The alignment was performed using HISAT2 through Genialis (https://www.genialis.com) with hg19-ERCC as reference. Normalization, differential expression, hierarchical clustering, and Gene Ontology analysis were then performed using RNA-seq analysis pipeline in Partek® Genomics Suite (Partek, St. Louis, MO, USA). Statistical significance was determined by ANOVA which is built into the Partek® Genomics Suite RNA-seq analysis pipeline. The significantly differential expressed genes (fold change >2 and false discovery rate <0.05) were then loaded into Ingenuity® Pathway Analysis (IPA) (Qiagen, Hilden, Germany) for upstream regulator prediction. Gene Set Enrichment Analysis (GSEA) program (Broad Institute) for pathway enrichment analysis was used according to the developer's instructions.

### RNA-seq Validation by NanoString®

NanoString (Seattle, WA, USA) human WNT nCounter panel was run using one OI type III and one control sample with RNA-seq data for the validation of gene expression fold change detected. The analysis was performed using nSolver™ under regular module. Genes with read count below 20 were considered as background and were removed from further analysis. Total 155 genes were validated. The consistency was determined by the percentage of genes that showed the same change in expression directions.

### Reverse Phase Protein Array (RPPA)

The RPPA was conducted by using a standardized protocol (Marini et al., ibid). Each sample was assayed in 3 technical replicates to account for technical variation. The protein expression intensity (PEI) of each biological sample was first derived from calculate the mean intensity of the technical triplicate of each biological sample. The average expression intensity of control group or type III OI group were then calculated from the mean of each sample's PEI in the group. The Student's t-test was used to determine the statistical significance between control and type III OI. Nominal P-value of 0.05 was used to determine significance.

### Western Blotting (WB)

50 μg of total protein was used for loading. After separation by SDS-PAGE gel, and transfer to PVDF membrane (MilliporeSigma, Burlington, MA, USA), 5% milk was used to block the membrane followed by overnight incubation with primary antibody (phospho-SMAD2 (3108S, Cell Signaling), SMAD2 (5339S, Cell Signaling) and GAPDH (G9295, Sigma-Aldrich) in 4° C. Signals were captured using ChemiDoc™ Gel Imaging System (Bio-Rad, Hercules, CA, USA) after appropriate secondary antibody (Bio-Rad).

### Immunohistochemistry (IHC)

After deparaffinization, sections were incubated in 37° C. with 0.05% trypsin for antigen retrieval following 3% hydrogen peroxide treatment. After blocking with 5% normal goat serum, sections were incubated with primary antibodies of phospho-SMAD2 (44-244G, ThermoFisher Scientific, Waltham, MA, USA) overnight at 4° C. as per manufactures' instructions. Anti-rabbit secondary antibody (Vectastain ABC system, Vector Laboratories, Servion, Switzerland) was applied and blots were developed using 0.1% 3, 39-diaminobenzidine.

### Example 1: Disorganized Woven Bone and Increased Osteocyte Density in Bones from Children with OI Type III

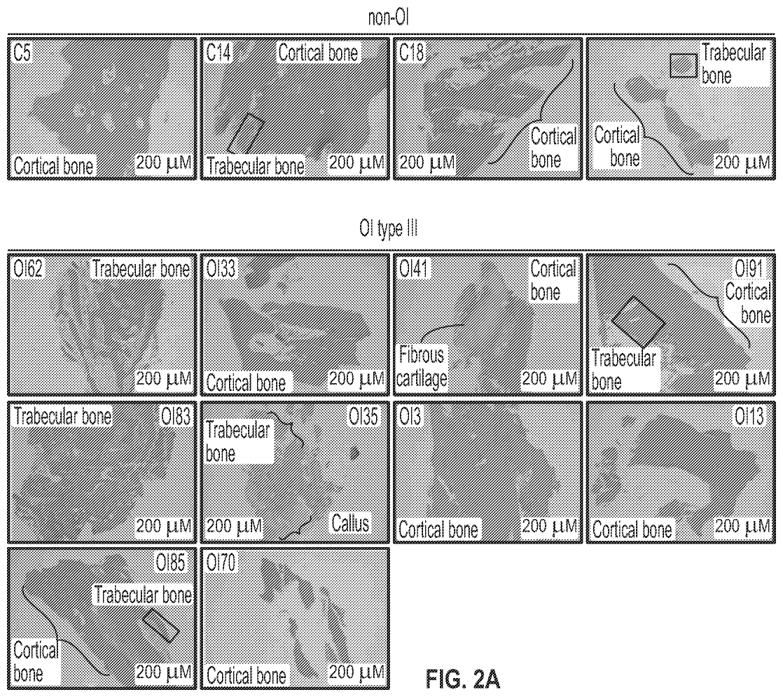

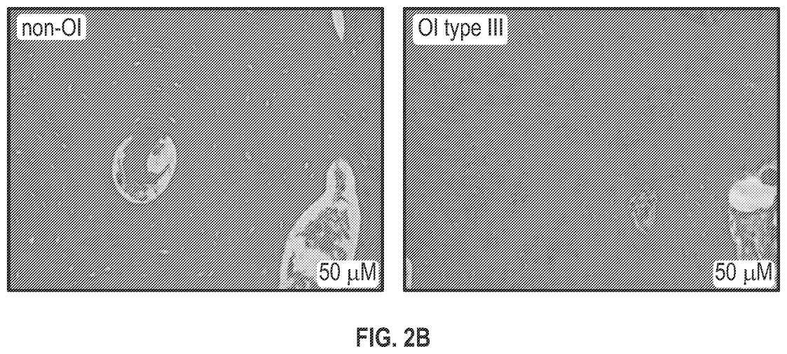

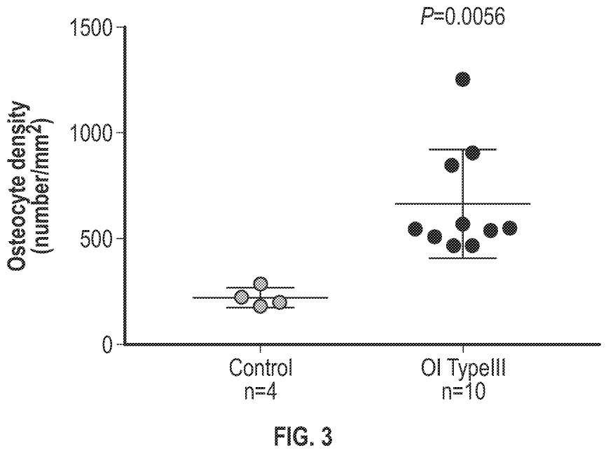

Bone fragments from tibia or femur were collected from 10 children with OI type III (9 with glycine substitution mutations in COL1A1 or COL1A2 and 1 with valine deletion in COL1A2) and 4 children who were not affected by OI (Table 1). Histologically, while the control specimens contained mostly cortical bones, OI specimens contained both cortical and trabecular bones. Morphological examination revealed that OI bones demonstrated disorganized Haversian system with predominantly woven bone compared to controls (FIG. 2). Consistent with earlier reports (Nijhuis et al., J Child Orthopaed. (2019) 13(1):1-11), osteocyte density was over 3-fold higher (686.44/mm² in OI vs. 221.94/mm² in control) in OI bone and the lacunae appeared more spherical compared to controls (FIG. 2 and FIG. 3). These data, which are typical for OI, allude to the high-quality of the samples.

### Example 2: Global Transcriptomic Analysis Demonstrates Increased TGFβ Signaling as Key Dysregulated Pathway in OI Type III Bone

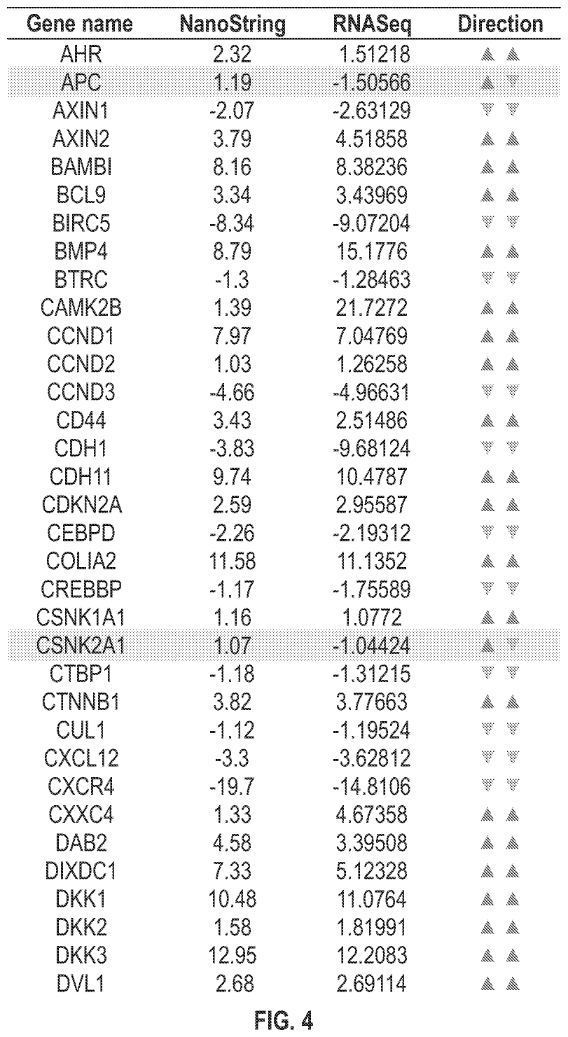

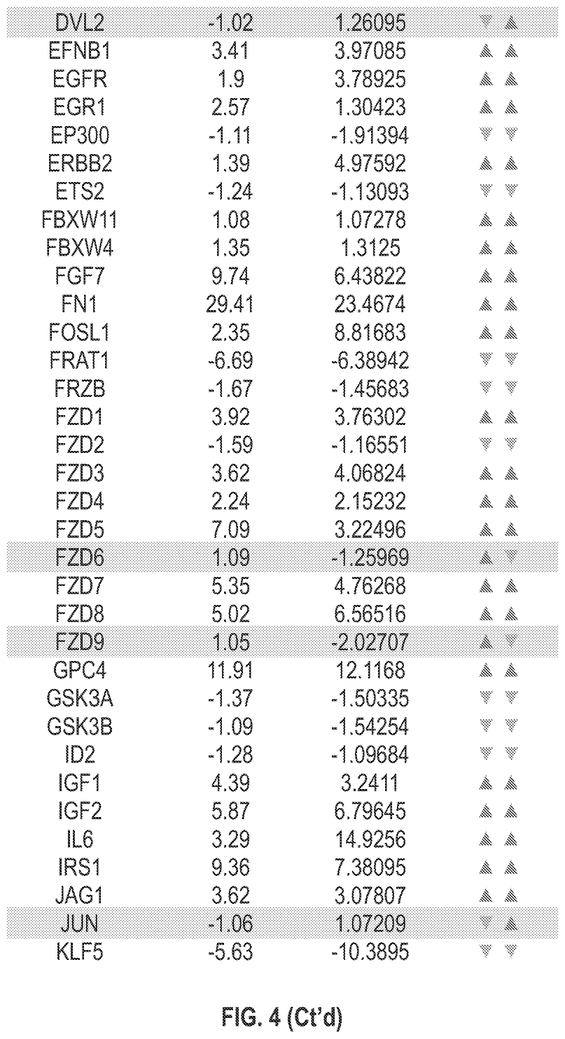

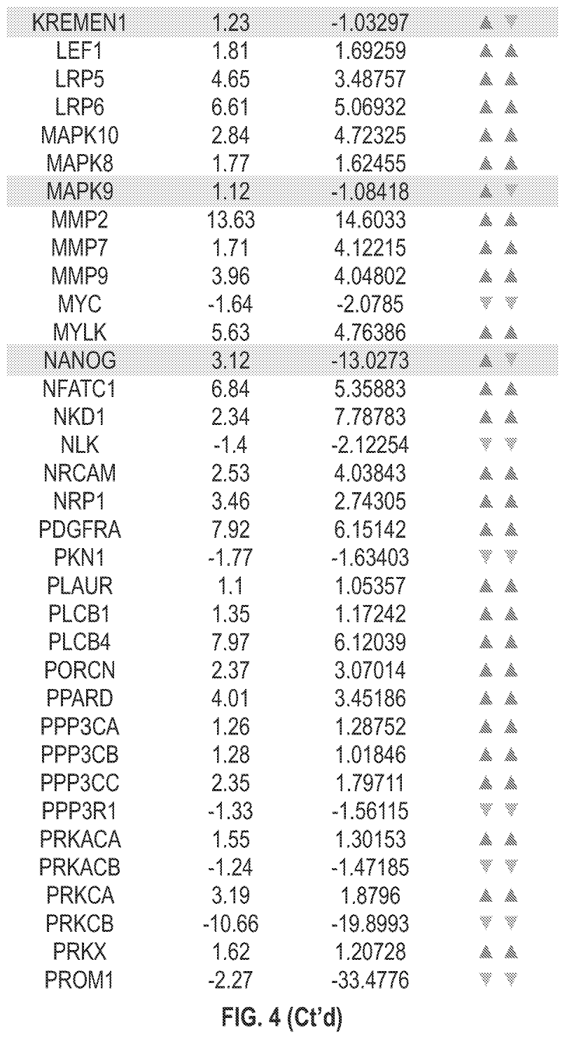

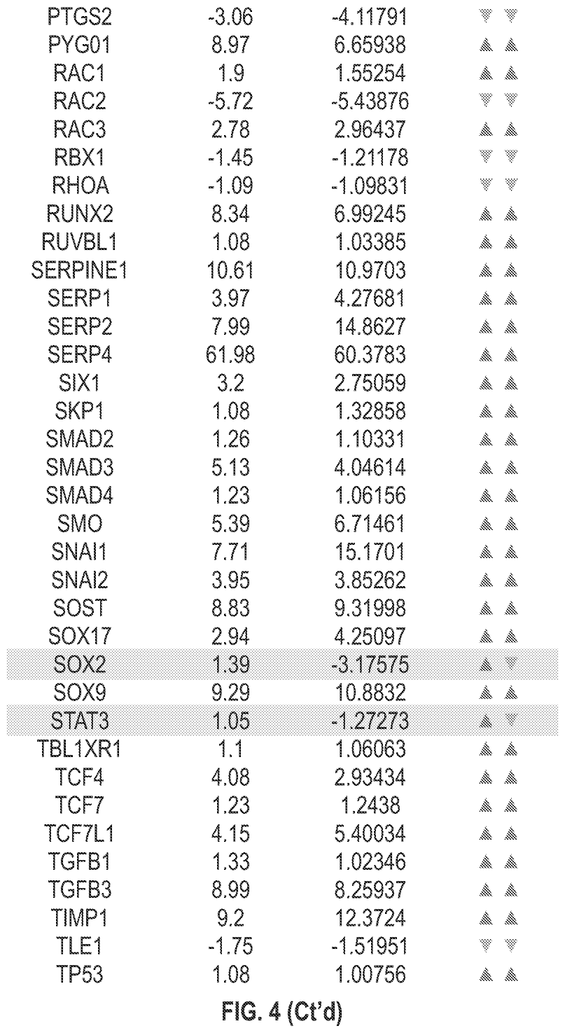

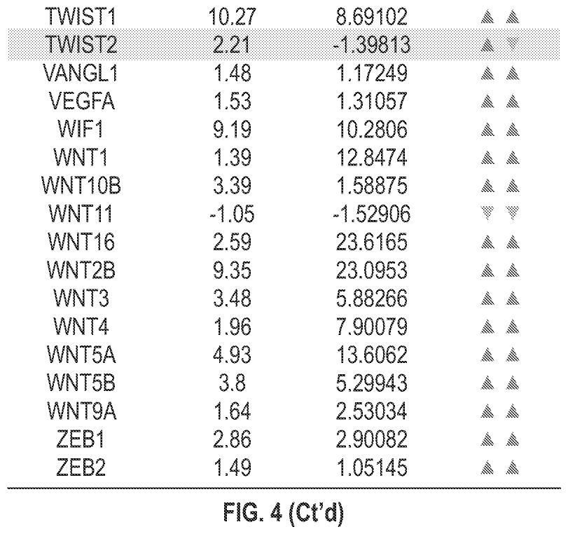

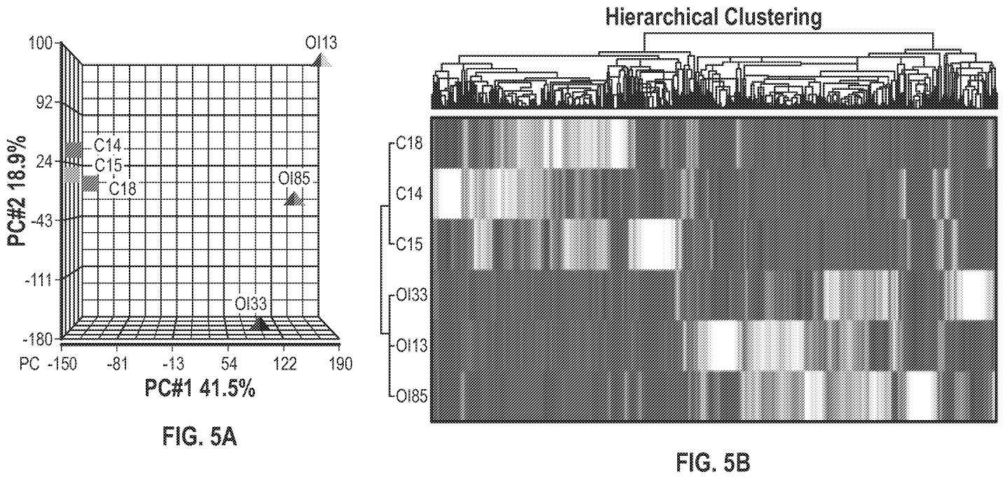

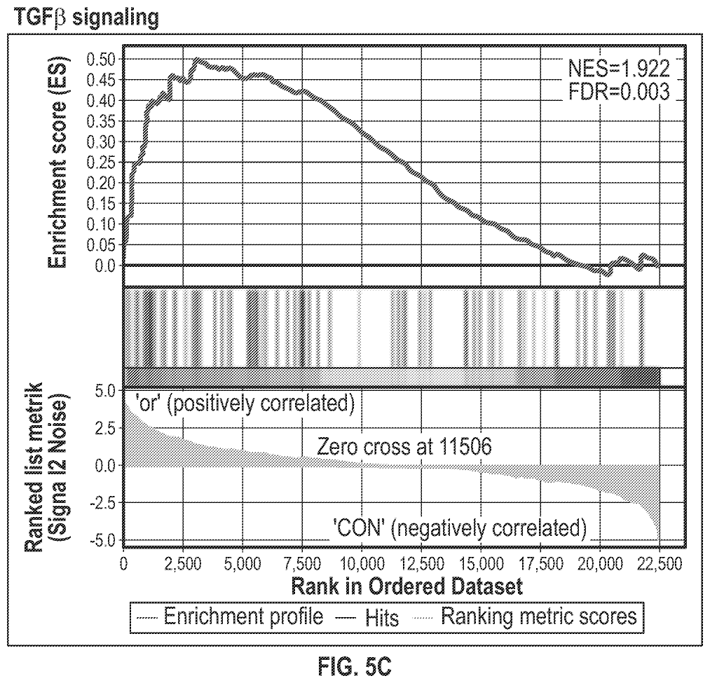

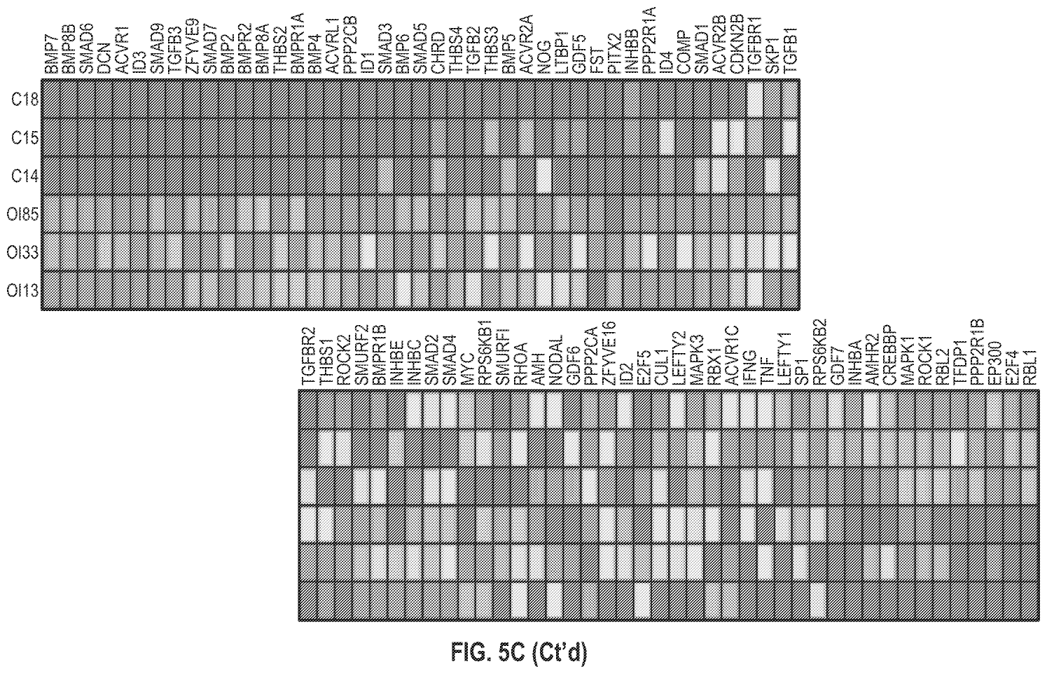

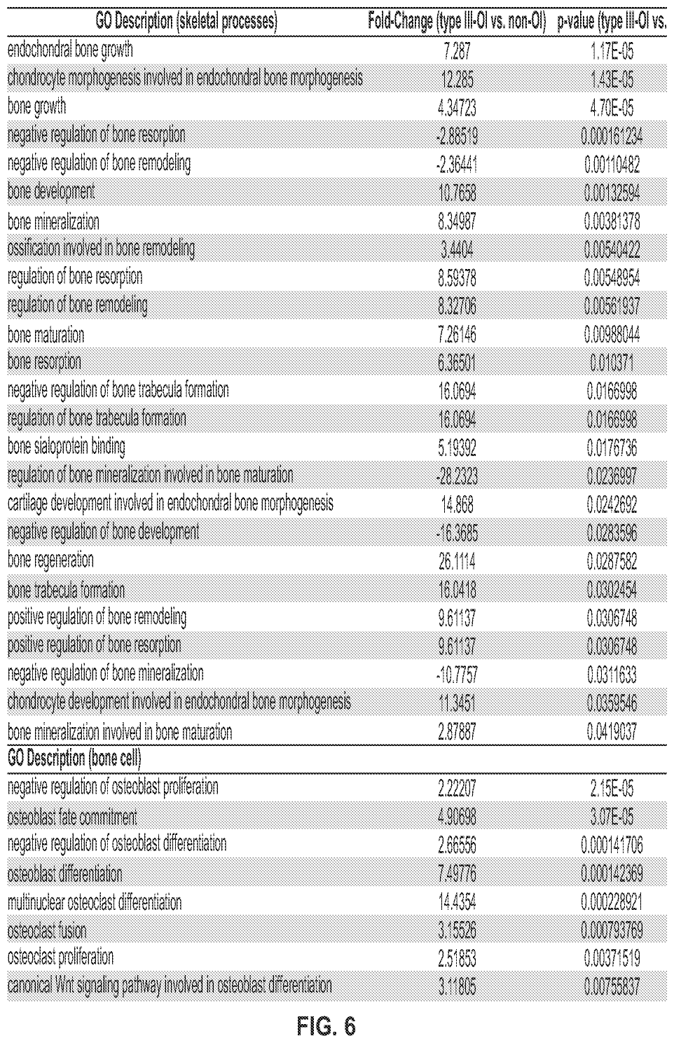

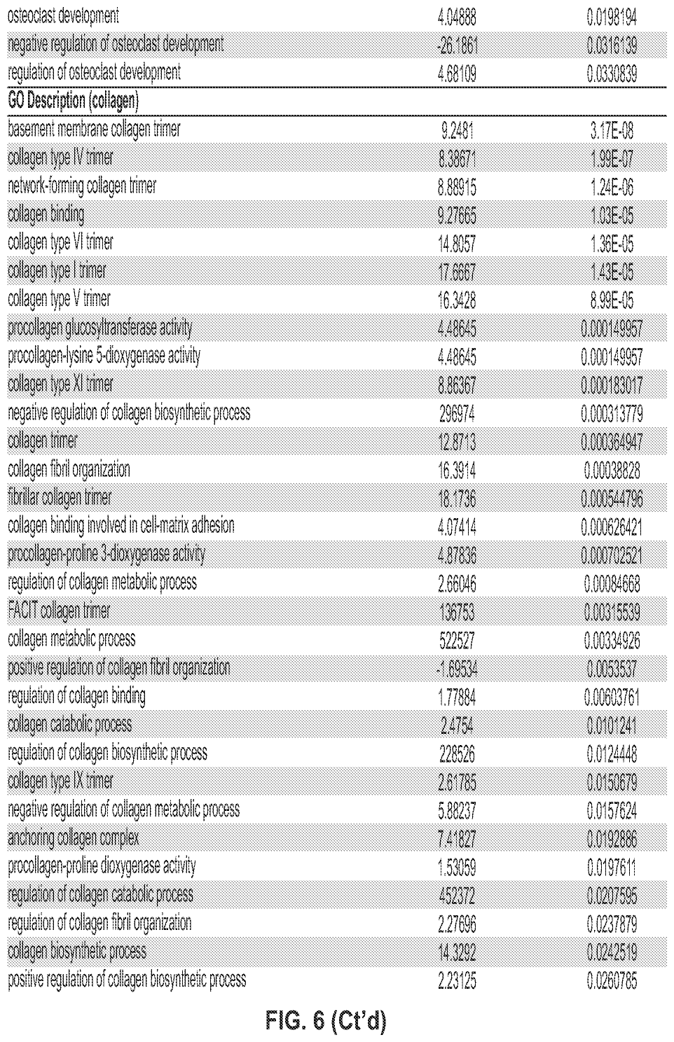

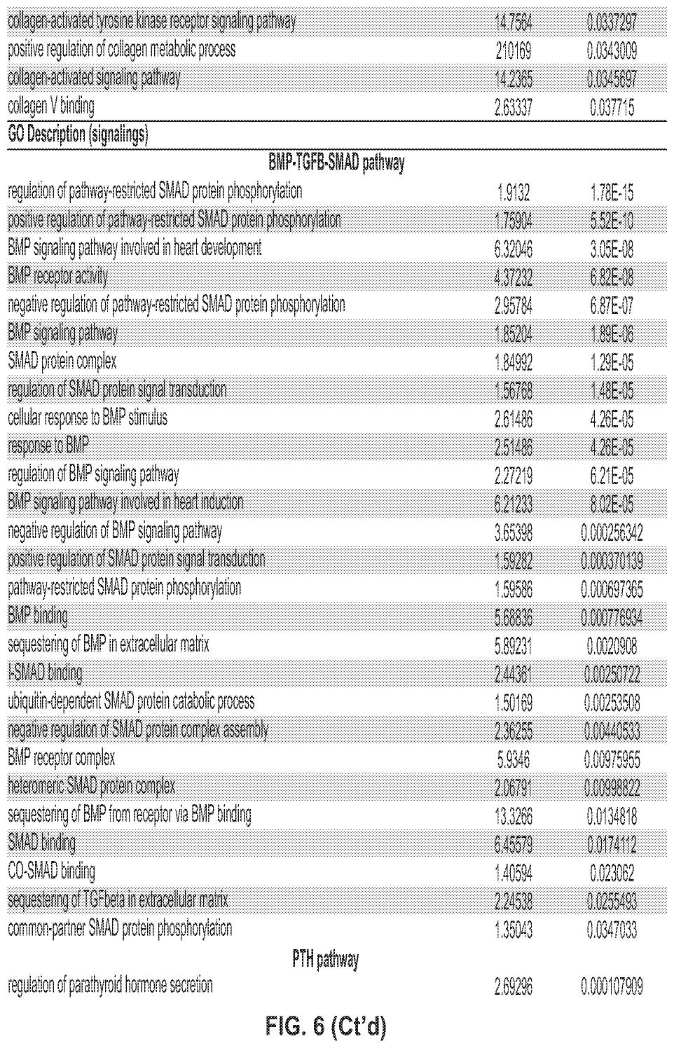

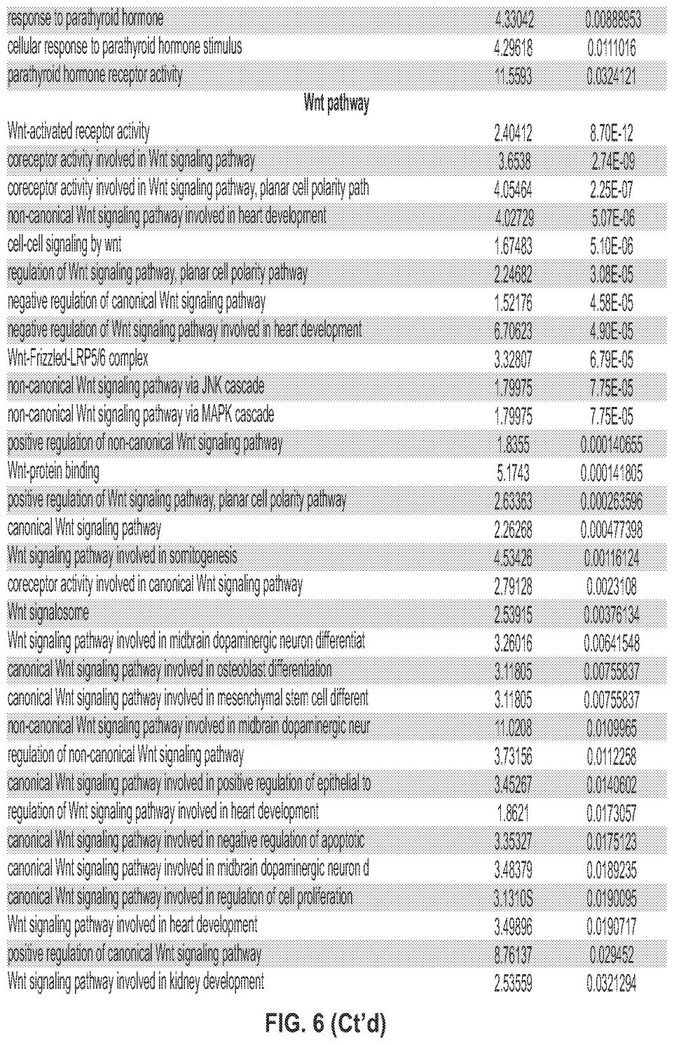

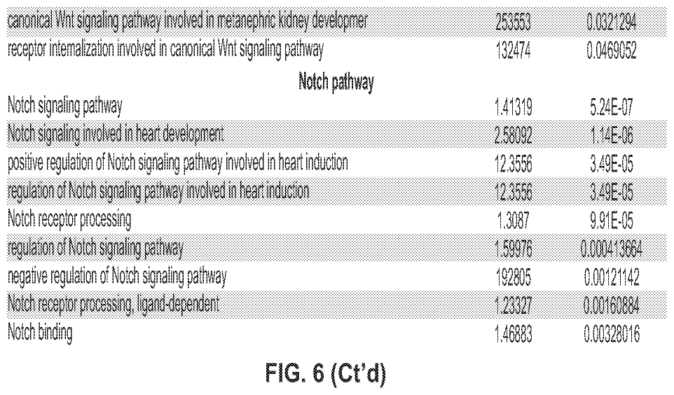

To identify the key dysregulated pathways in human OI bones in unbiased fashion, we performed RNA-seq using control and OI type III bones. To assure the accuracy of RNA-seq results, we validated 155 gene by NanoString® and demonstrated a consistency of 92% for expression fold change (FIG. 4). Principle component analysis (PCA) of transcriptomic data revealed a clear separation between control and OI bones (FIG. 5, Panel A). The gene expression profile was more homogeneous in control than OI bones. Hierarchical clustering of whole transcriptome RPKM showed distinct clusters between control and OI bones which indicated a change in overall molecular signature in OI (FIG. 5, Panel B). Following differential gene expression analysis, GO enrichment analysis was performed. In GO biology processes, the significantly enriched skeletal-related functions captured the molecular signatures of OI including increased bone formation, resorption, and remodeling along with decreased bone trabecula and bone maturation. Consistently, in GO cellular processes, increased osteoblast and osteoclast differentiation was identified in OI. In GO signaling pathways, major bone remodeling pathways including BMP-TGFβ, parathyroid hormone pathway, WNT, and Notch signaling were upregulated. Most interestingly, SMAD protein phosphorylation regulation was the most significantly upregulated pathway among all (P=1.78×10⁻¹⁵) (FIG. 6). These enrichment results are not only consistent with the known molecular pathological characteristics of OI bone but also unbiasedly revealed that a TGFβ downstream event, SMAD phosphorylation, was the most affected target in OI type III bone.

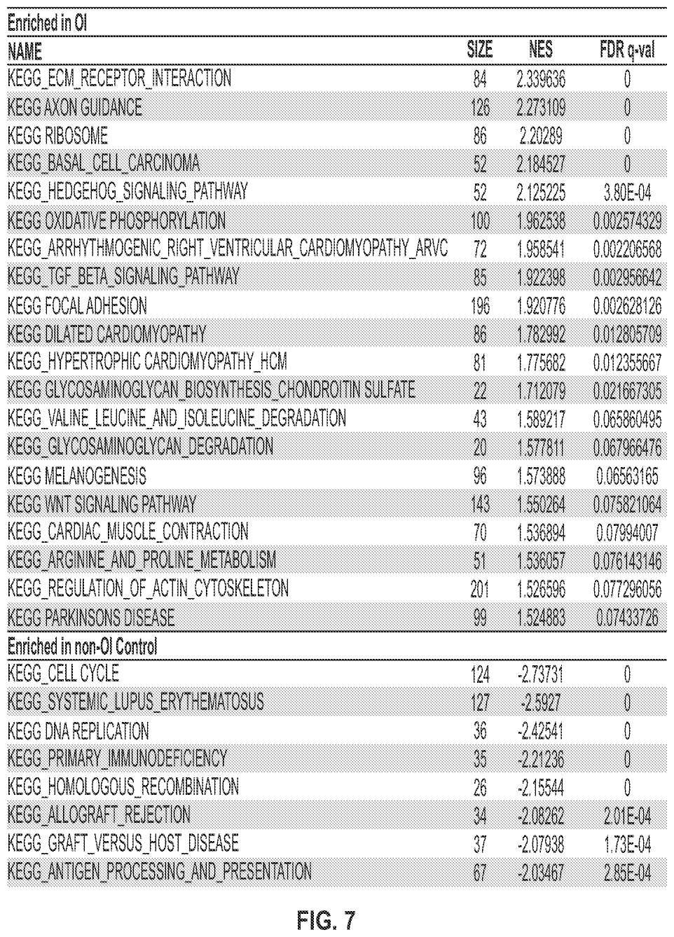

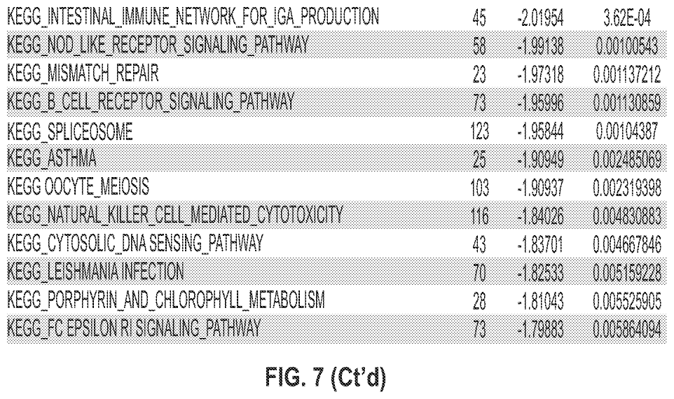

To independently explore change of signaling in OI bone, we next performed GSEA (Subramanian et al., PNAS (2005)₁₀₂(43):15545-50) using RPKM from control and OI bones. GSEA identified TGFβ signaling as a top significantly activated pathway in OI (FIG. 5, Panel C; FIG. 7). Together, these results suggested that TGFβ signaling is a major and the most consistent activated pathways in OI type III bone.

### Example 3: TGFβ is an Active Upstream Regulator in Bones from OI Type III

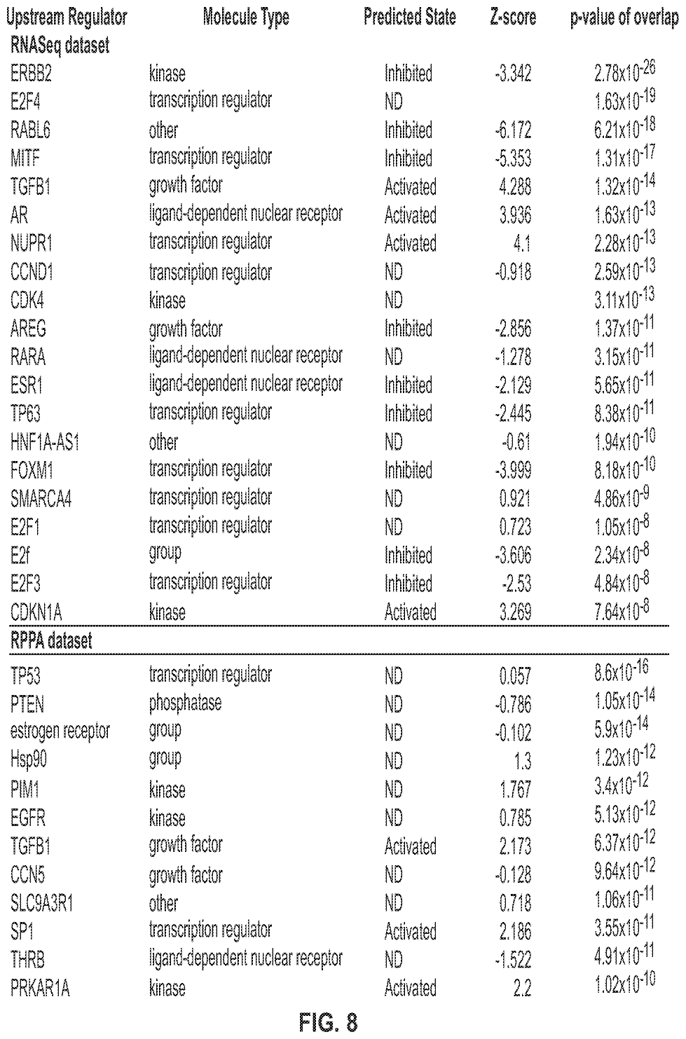

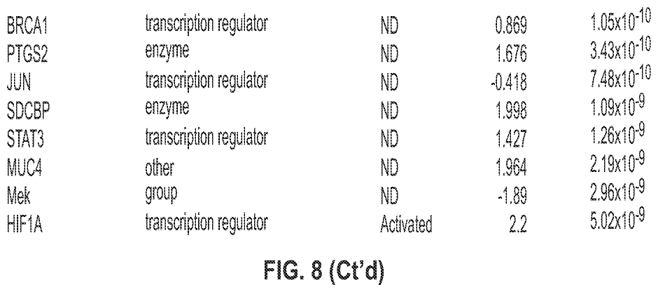

To investigate whether the activation of TGFβ pathway identified from transcriptomic analysis was indeed due to TGFβ ligand, we utilized IPA for upstream regulator prediction based on 3,722 significantly changed genes. Among all potential upstream regulators, TGFβ was the most activated upstream regulator identified (Z-score=4.28, P=1.32×10⁻¹⁴) (Table 2). To ensure these transcriptomic findings resulted in changes at the protein level, we performed targeted proteomic RPPA using proteins extracted from the bones. Among the 230 proteins examined, 29 proteins that showed nominal statistically significant intensity change between OI and control bones (FIG. 8). In this analysis, TGFβ was identified as the most significantly activated upstream regulator by IPA (Z-score=2.17, P=6.37×10-12) (Table 2).

TABLE 2

| | | | |
|---|---|---|---|
| Upstream Regulator | Predicted State | z-score | P-value |

RNASeq

| | | | |
|---|---|---|---|
| TGFβ1 | Activated | 4.288 | 1.32 × 10⁻¹⁴ |
| NUPR1 | Activated | 4.1 | 2.28 × 10⁻¹³ |
| AR | Activated | 3.936 | 1.68 × 10⁻¹³ |
| RABL6 | Inhibited | −6.172 | 6.21 × 10⁻¹⁸ |
| MITF | Inhibited | −5.353 | 1.31 × 10⁻¹⁷ |
| FOXM1 | Inhibited | −3.999 | 8.18 × 10⁻¹⁰ |

RPPA

| | | | |
|---|---|---|---|
| TGFβ1 | Activated | 2.173 | 6.37 × 10⁻¹² |
| SP1 | Activated | 2.186 | 3.55 × 10⁻¹¹ |
| PRKAR1A | Activated | 2.2 | 1.02 × 10⁻¹⁰ |
| PTEN | ND | −0.786 | 1.05 × 10⁻¹⁴ |
| estrogen receptor | ND | −0.102 | 5.09 × 10⁻¹⁴ |
| CCN5 | ND | −0.128 | 9.64 × 10⁻¹² |

### Example 4: Increased SMAD2 Phosphorylation in OI Type III Bone

To conclusively show the increased TGFβ signaling in situ in OI type III bone, we performed IHC using antibody against TGFβ downstream target, phosphorylated-SMAD2 (pSMAD2).

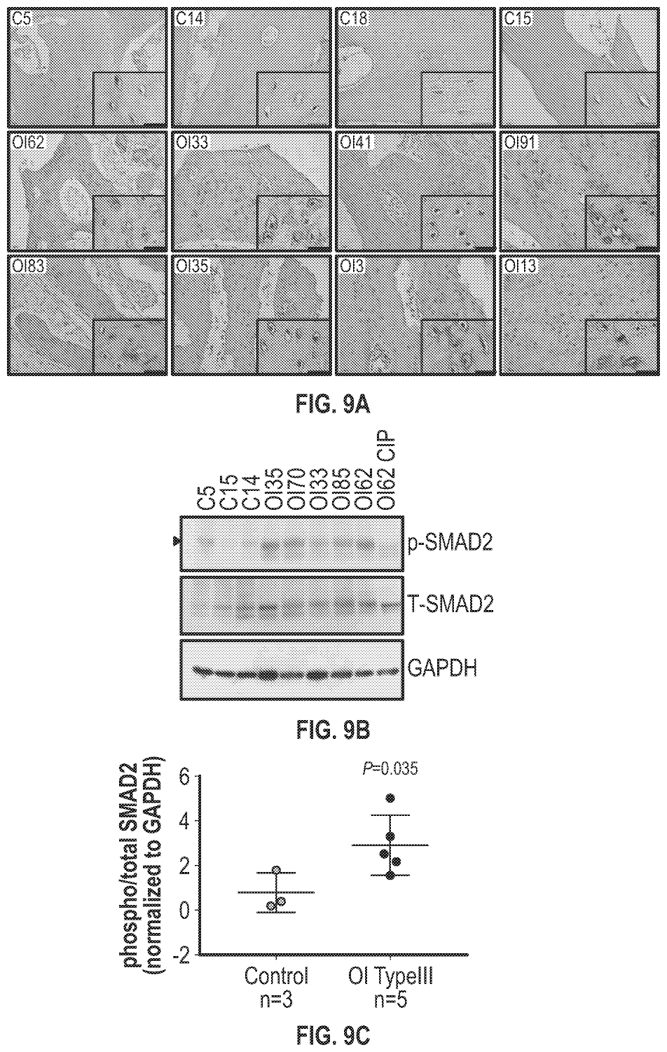

Compared to controls, we found a consistently increased pSMAD2 staining in OI type III bone (FIG. 9, Panel A). Through WB using protein lysate from control and OI type III bones, we observe an increase in pSMAD2 in all OI samples examined (FIG. 9, Panel B). Further quantification demonstrated a significant increase in the pSMAD2/total SMAD2 ratio in OI bone (FIG. 9, Panel C). All together, these results suggested that TGFβ activation is the driving pathogenic mechanism in humans with OI.

### Example 5: Fresolimumab as a Therapeutic Intervention in OI

To translate these findings, we conducted a phase I clinical trial evaluating the safety of fresolimumab, a human IgG₄ kappa monoclonal antibody, that neutralizes all mammalian isoforms of TGFβ. To be consistent with the preclinical and human data we have generated, only individuals with clinically moderate-to-severe OI caused by glycine substitution mutations in COL1A1 or COL1A2 or biallelic pathogenic variants in CRTAP, PPIB, or LEPRE1 were enrolled. A total of 8 individuals were enrolled (Table 3).

TABLE 3
A. 1 mg/kg dose cohort

| | | | | | Number of fractures |
|---|---|---|---|---|---|
| | | | OI | | sustained during |
| Participant | Gender | Age in yr | type | Causative pathogenic variant | lifetime |
| BCM222 | F | 25 | IV | COL1A2 p.Gly349Cys | 40 |
| FR003 | M | 47 | IV | COL1A2 p.Gly1078Asp | 25 |
| FR005 | F | 32 | III | COL1A1 p.Gly299Asp | >200 |
| BCM253 | M | 18 | VIII | LEPRE1 C.1244 1245insT and | >200 |
| | | | | c.1914 + 1 G > A in LEPRE1 | |

B. 4 mg/kg dose cohort

| | | | | | |
|---|---|---|---|---|---|
| FR007 | F | 36 | IV | COL1A1 p.Gly305Ser | 50 |
| FR009 | F | 27 | IV | COL1A2 p.Gly238Ser | 30 |
| FR011 | M | 28 | IV | COL1A2 p.Gly775Glu | 80 |
| FR012 | F | 55 | III | COL1A2 p.Gly655Glu | >100 |

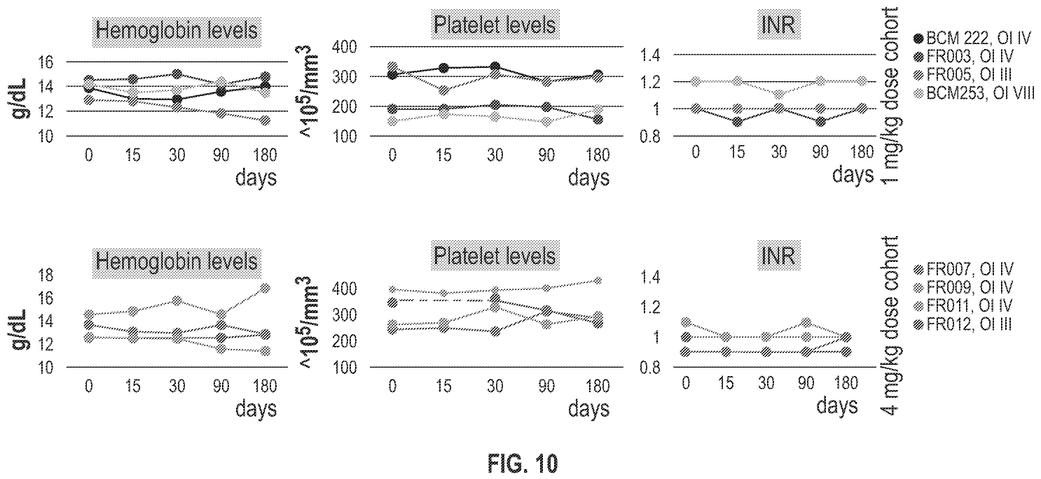

Four received single administration of fresolimumab at a dose of 1 mg/kg body weight and four received of 4 mg/kg body weight. Treatment with fresolimumab was well-tolerated. There were no serious adverse events (AEs) in both cohorts and no clinically significant laboratory changes were observed (FIG. 10). In the 1 mg/kg cohort, only two AEs were graded as being possibly/probably related to the study medication: nausea following administration of the drug and epistaxis. In the 4 mg/kg cohort, 7 AEs were graded as being possibly/probably related to the medication: malaise, headache, epistaxis, occult blood in urine, bleeding from skin scab, and corrected QT interval of 457 ms on day 180 in one participant.

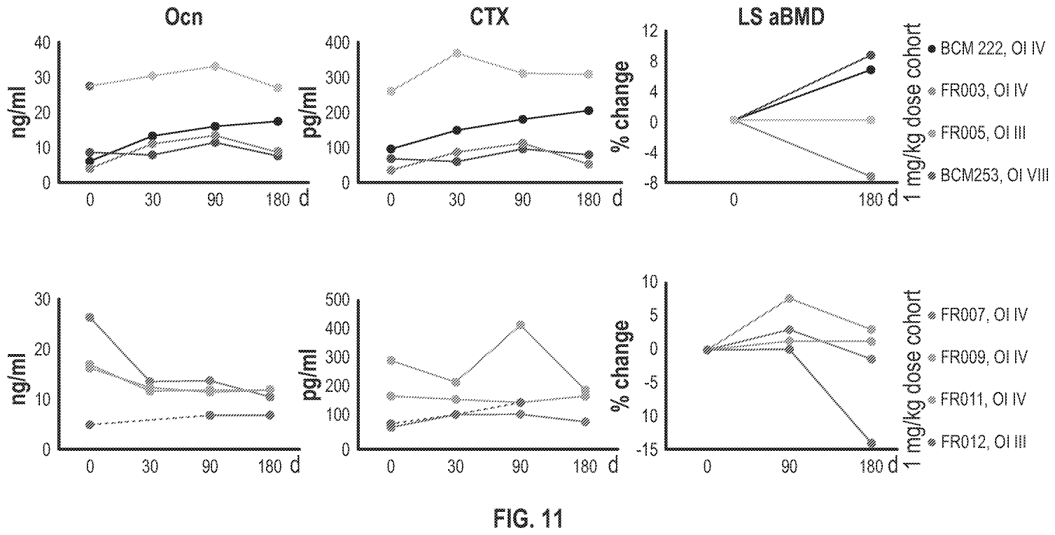

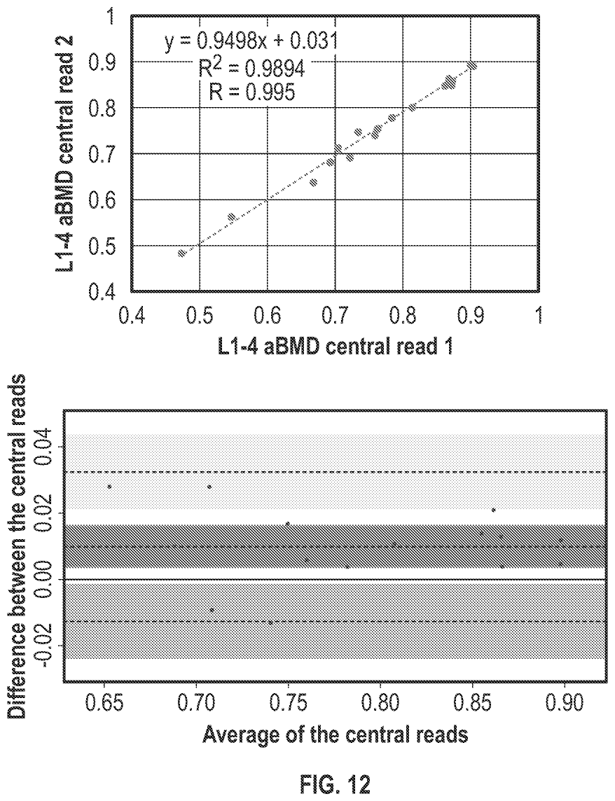

Treatment with 1 mg/kg of fresolimumab was associated with increase in markers of bone turnover, Ocn and CTX. The peak increase in bone remodeling was observed between day 30 and 90 post-treatment. Treatment with 4 mg/kg dose was associated with a sustained decrease in Ocn starting from day 30 after treatment (FIG. 11). The two central reads of LS aBMD (masked to time point of measurement) had a high degree of correlation (r=0.995) and agreement (FIG. 12). Thus, average aBMD from the two reads were used in calculating the percentage change from the baseline. With 1 mg/kg dose, two OI type IV participants showed robust increase in LS aBMD while the individual with OI VIII did not demonstrate any change. The participant with OI type III who showed a drop in aBMD had significant scoliosis that posed a challenge for while analyzing the results from the DXA scan.

In the 4 mg/kg cohort, LS aBMD was assessed at day 90 and 180. Two participants with OI type IV had a robust increase in aBMD by day 90. The individual with OI type III individual who showed a sharp aBMD drop sustained a femur fracture resulting in prolonged immobility; the aBMD was measured at a remote facility making comparisons less than ideal.

To conclude, we comprehensively and in unbiased fashion examined the global signaling abnormalities in OI, a Mendelian form of osteoporosis, in the present study. The findings and “omic-scale” data not only impact the treatment for OI but could also be relevant to other disorders of low bone mass. Furthermore, this study led to the surprising finding that treatment with even a single dose fresolimumab was associated with changes in bone turnover and aBMD at day 90 and 180 in our study. In addition, the unique feature of bone biology offers potential safety advantages due to lower cumulative dosage and administration frequency allowing for reduction of systemic toxicity. Indeed, the fresolimumab doses administrated herein were lower compared to those given in trials for melanoma, idiopathic pulmonary fibrosis, systemic sclerosis, and focal segmental glomerulosclerosis. With single dose administration, no serious AEs were observed. Furthermore, we observed different effect on bone turnover markers between the two trial doses. At 1 mg/kg dose, fresolimumab was associated with a mild increase in bone remodeling. However, 4 mg/kg fresolimumab treatment resulted in sustained suppression of bone turnover as shown by plasma Ocn levels. The effects on LS aBMD were more variable, depending on disease severity. Two participants with OI type IV had increases of 6.8% and 8.6% in LS aBMD with 1 mg/kg single dose. In 4 mg/kg cohort, one participant had a 7.6% increase and two showed increase of 2.9% and 1.3%, 3 months after infusion. These increases are higher compared to the anabolic agent teriparatide which demonstrated a 2% increase at 6 months in individuals with mild but not severe OI and is comparable to monthly high-dose setrusumab which was associated with a 5.4% in LS aBMD increase over 6-month trial period in OI types III and type IV (Eric et al., JBMR Plus (2021) 5(S1):Supplement:e10455). Two participants with OI type III (FR005 and FR012) had decrease in aBMD.

### SEQUENCES

The table below shows the amino acid sequences referred to in the present disclosure.

| SEQ ID NO | Description |
|---|---|
| 1 | Fresolimumab heavy chain |
| 2 | Fresolimumab or Ab1 light chain |
| 3 | Ab1 heavy chain |
| 4 | Fresolimumab or Ab1 HCDR1 |
| 5 | Fresolimumab or Ab1 HCDR2 |
| 6 | Fresolimumab or Ab1 HCDR3 |
| 7 | Fresolimumab or Ab1 LCDR1 |
| 8 | Fresolimumab or Ab1 LCDR2 |
| 9 | Fresolimumab or Ab1 LCDR3 |
| 10 | Fresolimumab or Ab1 V_{H} |
| 11 | Fresolimumab or Ab1 V_{L} |
| 12 | Human IgG₄ constant region |
| 13 | Human κ light chain constant region |
| 14 | D10 amino acid sequence |

1. A method for treating osteogenesis imperfecta (OI) in a human subject in need thereof, comprising administering to the subject a therapeutically effective amount of an anti-TGFβ antibody or an antigen-binding fragment thereof, wherein the antibody or antigen-binding fragment comprises
heavy chain complementarity-determining regions (CDRs) 1-3 comprising SEQ ID NOs:4-6, respectively, and
light chain CDR1-3 comprising SEQ ID NOs:7-9, respectively,
wherein the therapeutic effective amount is 1-10 mg/kg.

2. The method of claim 1, wherein the antibody or antigen-binding fragment comprises a heavy chain variable domain comprising SEQ ID NO:10 and a light chain variable domain comprising SEQ ID NO:11.

3. The method of claim 1, wherein the antibody comprises a human IgG₄ constant region and/or a human κ light chain constant region.

4. The method of claim 3, wherein the human IgG₄ constant region comprises a S228P mutation (Eu numbering).

5. The method of claim 3, wherein the antibody comprises a heavy chain comprising SEQ ID NO: 1 and a light chain comprising SEQ ID NO:2.

6. The method of claim 3, wherein the antibody comprises a heavy chain comprising SEQ ID NO:3 and a light chain comprising SEQ ID NO:2.

7. The method of claim 1, wherein the antibody comprises a bone-targeting moiety, optionally wherein the bone-targeting moiety is a poly-arginine peptide.

8. The method of claim 7, wherein the antibody comprises one or more poly-arginine peptides.

9. The method of claim 8, wherein the antibody is fused to a poly-arginine peptide at the N-terminus, or the C-terminus, or both termini, of the heavy chain, and/or at the C-terminus of the light chain, of the antibody or antigen-binding fragment.

10. The method of claim 7, wherein the poly-arginine peptide is D10 (SEQ ID NO:14).

11. The method of claim 1, wherein the OI is moderate-to-severe OI or type IV OI.

12. The method of claim 1, wherein the human subject is an adult patient (≥18 years of age), or a pediatric patient (<18 years of age).

13. The method of claim 1, wherein the human subject has a mutation in a COL1A1 or COL1A2 gene, optionally wherein the mutation is a glycine substitution mutation in the COL1A1 or COL1A2 gene or a valine deletion in the COL1A2 gene.

14. The method of claim 1, wherein the administration improves a bone parameter selected from the group consisting of bone mineral density (BMD), bone volume density (BV/TV), total bone surface (BS), bone surface density (BS/BV), trabecular number (Tb.N), trabecular thickness (Tb.Th), trabecular spacing (Tb.Sp), and total volume (Dens TV).

15. The method of claim 14, wherein the bone parameter is lumbar spine areal BMD (LS aBMD), optionally wherein the LS aBMD increases by at least 1-10% after the administration relative to baseline level.

16. The method of claim 1, wherein the administration decreases bone turnover and/or osteocyte density, optionally wherein the decreased bone turnover is indicated by a decrease in serum CTX or an increase in serum osteocalcin (OCN).

17. The method of claim 1, wherein the therapeutically effective amount is 1 mg/kg.

18. The method of claim 1, wherein the therapeutically effective amount is 4 mg/kg.

19. The method of claim 1, further comprising repeating the administering step every month, every two months, every three months, every six months, every nine months, or every twelve months.

20. The method of claim 1, wherein the antibody or antigen-binding fragment is administered by intravenous infusion.

21. The method of claim 1, further comprising administering to the subject a bisphosphonate, parathyroid hormone, calcitonin, teriparatide, or an anti-sclerostin agent.

22. The method of claim 21, wherein the bisphosphonate is selected from alendronate, pamidronate, zoledronate, and risedronate.

23-25. (canceled)
